(19) United States
(12) Patent Application Publication
(10) Pub. No.: US 2018/0129278 A1
(43) Pub. Date: May 10, 2018

(54) Interactive Book and Method for Interactive Presentation and Receiving of Information

(71) Applicant: Alexander Luchinskiy, Bad Kreuznach (DE)

(72) Inventor: Alexander Luchinskiy, Bad Kreuznach (DE)

(21) Appl. No.: 14/810,438

(22) Filed: Jul. 27, 2015

(30) Foreign Application Priority Data
Jul. 27, 2014 (DE) 10 2014 010 881.3

(51) Int. Cl. G06F 3/01; G06F 3/0483; G06F 15/02; A61B 5/04

(52) U.S. Cl. CPC G06F 3/013; G06F 3/0483; G02B 2027/0178; A61B 5/04; G06F 15/0283

(57) ABSTRACT

A method for interactive intercommunication with a book is presented. Method provides a possibility to get accompanying materials, in particular comments, updates, related informations in any form, etc. dependently on both a currently read matter inside a book page, and an user's reactions on the read matters. Reactions can be both controlled by an user (like e.g. a deliberate information request), and non-controlled by an user (like e.g. an automatic information request based on an automatic analyse of user's eyes pupils motions, attention preferences, current facial expressions, or his current or permanent biological or emotional state. To provide mobility and higher effectiveness of the method, the requested accompanying materials or other variants of book edition are presented in a virtual eye-glasses-type display directly in front of the user's eyes, because presentation of the placed on a book page surface information is not useful or completely impossible in a small smartphone display.

### CROSS-REFERENCE TO RELATED APPLICATIONS

This patent application claims priority of the following previous German patent application: DE 10 2014 010 881.3 from Jul. 27, 2014. The other related patent application is: U.S. Ser. No. 14/138,066 from Dec. 21, 2013.

### STATEMENT REGARDING FEDERALLY SPONSORED RESEARCH OR DEVELOPMENT

Not Applicable

### REFERENCE TO SEQUENCE LISTING; A TABLE; OR A COMPUTER PROGRAM LISTING COMPACT DISC APPENDIX

Not Applicable

### BACKGROUND OF THE INVENTION

As it is known, books can be presented both on a permanent, material carrier and in a display of an electronic device. For this aim one uses the information-carriering symbols (e.g. alphabet types/prints, hieroglyphs, ideographic pictures, knots, acoustic signals, etc.), which are presenting an information.

The books are known, which are executed as a real material object, wherein the information-presenting symbols (e.g. types/prints, hieroglyphs, ideographic pictures, etc.) are permanently presented on a real material carrier (as for example, among other possibilities, on a paper, parchment, papyrus, plates, stone, etc.). Wherein an above-mentioned real material carrier is used for a not-changeable/permanent presentation of one piece of information (piece of symbols, in particular a text). These above-mentioned information—(symbols-, text-,)—carriers can be connected together to use a book more comfortable. As a most usual actual embodiment example of a real book with a real, material symbols-carriers one uses a pile of paper sheets, which sheets are connected together on one of their sides (normally on the left side, but sometimes also on right side or on the top side of the sheets). An other example of a real book with real, material information—(symbols-, text-)—carrier is roll book, wherein this carrier is winded around a cylinder.

In general this method of an information presentation can be characterised as a generally known method of information presentation by means of a real (not virtual) book or by means of other printed editions as for example real periodicals, journals and newspapers. These traditional books or typographical & printed materials do not give an user any possibility for an interactive processing of information (inclusively also a detailed actualisation of content and for updates), which possibility is provided by electronic devices. On the other hand a method of presentation of information by means of a computer, by means of electronic books (kindles) or by means of other electronic devices cannot completely displace books and printed editions, because they belong to the base constituents of the cultural traditions.

Besides, also other kinds of books are known, which books one read by means of other organs of sense as eyesight. There are in particular the books, which one reads by finger sense or the audio books.

Historically the books with knots-symbols are known, which are consisting of a horizontal cords-carrying rod and cords with knots, which cords are hanging on this rod. On some opinion one read the information from the knots not (or not only) by eyes, but with the tactile sense on fingers. A modern further development of this tactile-sense-based kind of books are the books for blind people with the Braille-type.

The electronic books (e-book, kindle) are known, which are executed as a real, material object comprising an electronic device with a display, wherein the information-presenting symbols are presented in this display, and wherein the electronic device comprises elements for manual control, in particular for changing of the book pages images or for enlargement-decreasing functions. Furthermore, in this case a display is a symbols carrier. In the surface plane of this symbols carrier the symbols are presented. This display is also a real object, as also a symbols carrying page in a case of a real book. But the symbols themselves are the virtual, changeable objects, which are presented in a display surface plane. Therefore many different symbols can be presented on the surface plane of a same display-symbols carrier in the different moments of time.

A book can be also presented by means of a tafel-PC (I-pad), laptop, and at all by means of a display of any PC or electronic device. In all cases of these book-presenting variants the book pages are presented in a display, and more concretely on a surface of this display, in a surface plane of this display. And these book pages images are always changed in this display after the user has read them. The main difference between any of these electronic devices are only control interfaces, which are different in a e-book (kindle), tafel-PC and laptop.

Furthermore, smartphones (in particular I-phone), as well as tafel-PCs (in particular I-pad) with an interactive display are generally known, which belong to state of technology. Also the usual portable devices, as for example mobile telephones or laptops with not-interactive displays and not-virtual keyboards are known.

As it is known, there are two mutually exclusive requirements to these devices:

On the one hand, for the better portability the whole device have to be as small as possible, and consequently, it's display have be small.

And on the other hand, the display have to be as large as possible, to contain more information, which one could be read and processed by an user. The same is true also for a keyboard (both for a physical one, and for a virtual keyboard in an interactive display), which keyboard on the one hand have to be as small as possible, but on the other hand this small keyboard have to be still usable. The same is true also for all possible kinds of control means, wherein the control is executed by hands or fingers.

A method is generally known, wherein an eye's position is watched by a webcamera (i.e. computer-connected videodevice), then this information is further transferred into I-pad (or in general—into a computer), there this information is processed, and finally the displayed at a display text is changed, when the eyes attain the end of the displayed text (Scroll-Function). This method, nevertheless, does not provide a possibility to use the opportunities of mutual interconnection of an user with a computer in a full measure.

### BRIEF SUMMARY OF THE INVENTION AND DETAILED DESCRIPTION OF THE INVENTION

Under the term “computer” one understands here all kinds of computers, or of devices, which execute the functions of a computer (in particular stationary PCs, laptops, I-pads, all kinds of smartphones or mobile telephones with computer functions, electronic chips, etc.).

Under the term “webcamera” one understands here all possible kinds of webcameras, videocameras or all kinds of other devices, which are possible to receive a visual information and further pass it to a computer.

Under the term “User-observing-webcamera” or “UO-webcamera” one understands here below an a.m. webcamera, which one observes (watches):
positions of eyes pupils of an user, in particular relative to a book page, or relative to definite areas or points in a book page, and/or:
positions of eyes pupils of an user, in particular relative to external field outside a book page, but inside a field of vision of the user, (in particular a real environment, which one lays outside the book pages images—in the embodiment example, wherein one uses virtual books); and/or
state and change of states of facial and facial expression muscles or gesticulation of an user. This webcamera is directed at the user's face. Therefore this webcamera can be fixed on a computer, on a book, on a table, etc. In particular the functions of this UO-webcamera can execute a permanently installed webcamera of a computer (or of a laptop, or of a smartphone, etc.), if this webcamera is directed at the user.

Under the term “Book-observing-webcamera” or “BO-webcamera” one understands here below an a.m. webcamera, which one observes (watches) a book, book pages, markings or codes on a book or on book pages, and in the necessary cases also the positions and motions of fingers tops of an user relative to the book page. This BO-webcamera is directed at the book and at the book pages. This BO-webcamera can be fixed, in particular, on an user (on his glasses, on his clothes, like on a shirt pocket, on a breast pocket, on a lapel, on a collar, etc.).

The fields of vision of an UO-webcamera and of an BO-webcamera are different, because these webcameras are directed towards the opposite directions. In the field of vision of an UO-webcamera an user is observed (a.o. his face or upper part of his body, a.o. his eyes pupils positions, his motions or gesticulations, or the similar). In the opposite, in the field of vision of an BO-webcamera a book and book pages are observed (a.o. the markings or codes on the book and on the book pages, and also, if necessary, the positions and motions of a finger top of an user relative to a book page, or motions of a top of an used by an user pointing agent, relative to a book page).

Under the term “real book” one understands here a real, material book with the real, material pages, in particular a “hard-cover” book, a paper-cover/soft-cover book, a comics, a journal, a periodical, a material symbols-carrier, which one is rolled up as a rouleau, or also others bound or not-bound printed editions. Nevertheless oftly it is shortly written “book” instead of the term “real book”, if it is clear obviously from the context, what is meant.

Under the term “e-book” and “kindle” one understands here the “e-Buch”/“Kindle” in an usual meaning.

Under the term “virtual book” one understands here a virtual “hänging in an air” image of a book or images of open book pages, which are located in a virtual space in the field of vision of an user, with which aim the user carries on the special glasses or contact lenses or other kind of optical devices in front of the eyes, wherein this device creates this virtual image in front of the eyes of the user, in some distance from the eyes and from the external surfaces of the glasses (or devices).

Some embodiment examples of the invention are described below.

Aim of the presented in the claims invention is to provide an interactive automatical processing of the presented in a book (or in other kinds of printed editions) contents. The invention makes it possible, to integrate an interactive intercommunication with a traditional real book (or printed edition) in one general scheme of intercommunication with a computer or also with a website.

Additional aim is to provide a more effective intercommunication with a computer or with a book, with a computer game or with a smartphone. This problem is solved in particular a) by a directly, through eyes and facial expression, but not only through fingers executed interactive interaction of an user with a computer; and b) by a simultaneous additional or alternative presenting of videoinformation directly in front of the eyes of the user in the form of a virtual (appearing/imaginary/“in the air hanging”) image (virtual image).

Besides the direct aims, the attained by this inventions advantages are, in particular, that it makes it possible to increase a speed and comfort of an interaction with a computer, and to realise the reactions of the computer on a biological or emotional state of an user, or also to provide a comfortable interactive communication of a user with a book or with any other traditional (not-electronical) printed edition.

In one embodiment example the webcamera (UO-webcamera) of a computer (PC, laptop, I-pad, smartphone or any other kind of computer) can watch
the dimensions (diameter) of the pupils of the eyes, and
the position of the pupils of the eyes relative to the display,
so that when an user fixes his look at any part of a display to see any details, the pupil of his eye decreases itself, the above mentioned UO-webcamera recognises (see) this decreasing and passes information about it to the computer, after that the computer processes this information and enlarges (increases) the above described detail of the picture in the display. (In further description the pupils of the eyes will be named as pupils).

This way the enlargement (increasing) or decreasing of a part of a displayed matter on a display is caused and controlled not by fingers, but by the dimensions of pupils (i.e. by the pupil muscles).

The same way one can cause and control the enlargement or decreasing of a part of a picture (displayed matter) on a display by the facial expression muscles. In particular one can screw his eyes up or blink by his eyes, and the UO-webcamera and the computer with a correspondent software can react on these movements of the parts of user's face.

In one embodiment-example of the invention the computer watches through the UO-webcamera the position of the eyes, passes this information in computer, after that this information is converted in the digital (electronic) form and processed, after that the computer changes the picture (displayed matter) on the display correspondently to this information, wherein simultaneously
a) the dimensions of the pupils or changes of the pupil's dimensions and
b) the position of the eyes or of the pupils relative to (with respect to) the display (a.o. relative to the virtual display, which one appears through display-spectacles)
are watched by the UO-webcamera and processed by the computer, and on the grounds of the results of this processing the correspondent part (segment) in the picture in the display (in the displayed matter) is enlarged or decreased.

To provide a recognition by a computer of the positions or of the changes of positions of the eyes or of the pupils of the eyes relative to the display, an user can firstly direct his pupils to (look at) at least two ponts (markings) in a display, wherein the positions of these points (markings) relative to other presented in a display visual information is known to the computer. This way the position of the eyes pupils relative to the display (i.e. where the user looks at) is adjusted.

In one embodiment-example of the invention the user directs his eyes or pupils (i.e. looks) at one definite point in the display, and he screws his eyes up or blinks by his eyes, the UO-webcamera and computer recognise these facial expression changes in the muscular system of the face, and the computer enlarges in the display this corresponding surface area of the displayed matter, at which surface area the user looks, or the computer stops the enlarging when this above mentioned eye screw-up muscle relaxes, or the displayed picture is decreased if one executes some determined facial expressions (face muscles states) on his face (in particular, one relaxes the eye muscles (one makes larger his eyes), one opens his mouth, or one uses any kind of other facial expression-forming movements of the face muscles, wherein a correspondence between the said facial expression-forming movements of the face muscles (or face muscle tensions) and the reactions of the computer can be adjusted beforehand; or an user can direct the eyes (look) at a displayed on a display enlargement-decreasing scale (line), then to focus the pupils of the eyes on a definite point of this scale, then to screw the eyes up or to blink by the eyes, and this way the computer recognizes through the UO-webcamera the preferred by an user resolution (grade of enlargement or decreasing), and the computer adjusts (set) this grade of enlargement or decreasing in a display.

In one embodiment-example of the method of the intercommunication with a computer, in particular for using in a computer game, the facial expression changes (tensions of the facial muscles) are watched by a computer through a UO-webcamera, this information is passed to the computer, after that this information is converted in a digital (electronic) form and processed, and after that the computer changes the displayed matter in it's display according to this information.

In one embodiment-example of the method of the intercommunication with a computer, in particular for using in a computer game, the enlargement/decreasing scale (line) appears in a display, an user directs the eyes (or pupils of the eyes) at a definite point of this scale, the user focuses the eyes (makes smaller the pupils of the eyes), or the user screws his eyes up or blinks by his eyes, or the user moves his lips, or he moves an other, beforehand “agreed” with the computer program (adjusted/set up), part of a face, and this way the computer recognizes through the UO-webcamera the preferred by an user resolution (grade of enlargement or decreasing), and the computer adjusted (set) this grade of enlargement or decreasing in a display, wherein also a marker (as for example a little quadrangle) can appear on the that point of the above mentioned scale, which point is selected by the user the above described way.

If the user changes the resolution, the above-mentioned marker glides along the scale to indicate the correspondent grade of the resolution. Besides, the above-mentioned scale can appear in that place (in the picture of a display), where to the eyes of the user are directed, and, correspondently, where the enlargement-decreasing mechanisms are launched, as it was described above. Or this scale can be placed at the margin of the displayed field near the margin of the computer display, and the user launches this above-described enlarging-decreasing mechanism by directing his eyes on this scale (at it's definite part) simultaneously with the focussing of his eyes (decreasing of the pupils). Or the user launches this above-described enlarging-decreasing mechanism when he directs his eyes on this scale (at it's definite part) and simultaneously he screws his eyes up (or blinks by his eyes), or makes movements by his lips, or he executes another, beforehand “agreed” with the computer program (adjusted/set up) movements of parts of his face (movements of the facial expression muscular system).

In one embodiment example of the invention the computer game (a system) comprises a lie-detector, wherein the computer game asks the user questions (verbally, i.e. with the words, or by proposing of different choices of verbal or motorical (motive), or both, reactions or situations in a game), and, dependently on his answer (or dependently on his above mentioned choice of reaction or situation in the game), in connection with the processed by the computer readings/indications of the lie-detector, the computer game choose a variant of the further behaviour (situation, reaction, program, velocity of actions, etc.) of the computer game with the user.

In one embodiment-example of the invention the computer game (the system) comprises an encephalograph, a myograph, or any other medical-diagnostic equipment, which equipment executes the reading of the current medical-biological parameters (in particular finally of the emotional state) of an user, watches these parameters and passes these data into the computer, after that these data are processed by a program of the computer game, and, dependently on the results of this processing, the computer game choose a variant of a further, either current in the moment or strategical, behaviour with the user (situation, reaction, program, velocity of actions, variants of the verbal (with the words) answers or communications, etc.).

In one embodiment-example of the invention a facial expression (state of face muscles) of an user is watched and analysed by a UO-webcamera and computer, and dependently on the results the computer changes the current reactions of the computer game on the actions of the user.

Also an emotional or biological state of an user can influence on a behaviour & actions of a personage (virtual actor/character) of a computer game. I.e. the computer processes the biological parameters of the user, and, dependently on the results, the computer game proposes the further parameters of the game (situations, reactions, speed, etc.). It gives a possibility to execute and chose the more medically safe, or the true way training, as well as the more intensive or the less intensive modes of game, dependently on the both permanent (as for example age, state of health, kind of temperament, aim of a game) and current (level of excitation, pulse frequency, etc.) parameters of the user.

In one embodiment example of the invention an UO-webcamera is fastened to an e-book (kindle) (or this UO-webcamera is fastened near the e-book (kindle) or/and these camera and e-book are electronically connected one with another) to provide a possibility to observe the changes of positions of the eyes pupils of the user, and to provide a possibility to observe the changes of facial expressions of the user. In this case the e-book comprises also a device, which one recognises an actual page of the e-book “currently to read” and passes this information into the computer. After that it is analysed by the computer in particular a) which changes of facial expressions of the user are generated by this actual page in general; b) where to exactly in this page the user looks at in the current moment of time; and c) which facial expression reactions are generated in this current moment of time. Correspondently, the computer presents in it's display the information, which one currently corresponds to the a.m. current positions of the eyes pupils, and to the current facial expressions or to the current changes of facial expressions.

In one embodiment example of the invention the device comprises:
an usual (not electronical) book,
an UO-webcamera (for observation of the user), which UO-webcamera is fastened to the book,
an other BO-webcamera (for identification of the book, of the book open page and it's contents), which BO-webcamera is fastened to the book or to the user (for example to his glasses or to his clothes),
a computer with a display, and
a website (i.e. means, which make possible a functioning of an internet-website).

Or instead of (or additionally to) the a.m. UO-webcamera for observation of an user, the book comprises a device, which one recognises, what point or area in a book page has the user touched by the top of his finger. And according to these recognition results the correspondent to this point or area information is presented in a display.

Under the term “book” one understands here in particular a “hard-cover” book, a paper-cover/soft-cover book, a comics, a journal, a periodical, or also others bound or not-bound printed editions.

Under the term “display” one understands here, additionally to the usual meaning, also a device, which one is placed in the glasses (spectacles) or in the eyes contact lenses, wherein, instead of a real image, a virtual (appearing/imaginary/hanging in an air) image is formed by this device, which virtual image appears immediate/direct in front of the user's eyes, in particular this displayed matter (image) is created/formed in the lenses of glasses (spectacles), or in the lenses cover pieces, or only in one of the two lenses cover pieces, or in the eyes contact lenses, or only in one of the two eye contact lenses.
Some embodiment-examples of invention provide a possibility to see a large picture with a displayed matter, which large picture is generated by a small smartphone (mobile telephon). Therewith the possibility is provided to play a game more comfortable, as well as to solve the contradiction between the portability of a mobile telephone and good sight-ability of a display of a mobile telephone. On the one hand a mobile telephone must be possibly small and portable, to make better it's portability and handiness. On the other hand the display of a mobile telephone must be possibly large, because a presentation of information on a big display is more useful. This problem is solved in the listed in the claims features. Furthermore, instead of—or additionally to a real material keyboard, the smartphone or mobile telephone can comprise a virtual keyboard, wherein, in particular, the movements of fingers (or movement of a finger) are recognised by a correspondent device (means), and simultaneously these movements or one movement are presented in the virtual display according to one of the claims. Therewith one can use this virtual keyboard by moving of fingers.

Normally a presenting of a visual information takes place by a forming/creating of a visual information on a display of a smartphone (mobile telephone). These signals normally cause a real picture (real image) on the smartphone display. Nevertheless additionally (or instead of it) these signals can be converted in an image, in particular in a virtual image (appearing/imaginary/“in the air hanging” image), which one appears directly (immediate) in front of the user's eyes, in particular this displayed matter (image) is created/formed in the lenses of glasses (spectacles), or in the lenses cover pieces, or only in one of the two lenses cover pieces, or in the eyes contact lenses, or only in one of the two eyes contact lenses.

In one group of the embodiment examples an interactive (intercommunication-able) book is realised.

In one of these embodiment examples the method is executed as follows:

One coats an automatically readable graphical or electromagnetical code-marking on a book. These code-markings can a.o. be printed on a book still on the stage of a book producing, simultaneously with the book contents printing. Or these code-markings can be sticked.

It can be a.o. the following three below described kinds of markings (1)-(3):

(1) “General Identification Code-Marking”.

This marking is coated a.o. on a cover or on a dust-cover (or on an upper page, or on a first page, or on a title-page, or the similar) of a book. This general identification code-marking can be coated on a book in many places of this book. Nevertheless only one single marking (for example on a dust-cover) is actually enough. A role of this marking can play any kind of an optically read-out-able by a machine code (as for example the known points-code, or dash-code, etc.), or an electromagnetically read-out-able code. Through this general identification code-marking the book is automatically identified by a system “webcamera, computer and website”.

(2) “Code-Markings for Each Page”.

These (not identical) markings are coated on each page. Each of these read-out-able by a machine markings identifies every page of a book. After the identification of the each book page by the system “webcamera, computer and website”, the information from the website, which one (information) belongs to this book page, is presented in a computer display.

(3) “Submarkings for Matters Inside Pages”:

By means of these markings the blocs of contexts inside a printed page of a book are marked. (Examples of these blocs of contexts are the separate words or sentences, pictures or parts of pictures, etc.). These markings can be presented or permanently printed in a book page in particular as a blue colouring and underlining of the marked words and sentences, and as a little fist with a forefinger at a picture, as it usually takes place when one electronically presents an information in a display. The system “webcamera, computer and website” identifies firstly a book by means of the a.m. “general identification code-marking”. After that the actual open page of this book is identified by means of the “code-markings for each page”. And after that the actual “submarking for matters inside pages” is identified, i.e. the marking, where to the user actually points by his finger top or by his eyes pupils position. And after that the information (data), which correspond to this marking, are found in the website and presented in the computer display.

Wherein an identification of an each “submarking for matters inside pages” can take place the following way: the a.m. computer identifies by the a.m. BO-webcamera the position (the coordinates) of finger tops of an user relative to the actually open book page surface. As this open page of the book was already identified through the “code-marking for each page”, the computer calculates, to which of the located in this page “submarkings for matters inside pages” concretely corresponds this position of the finger top.

In this embodiment variant the BO-webcamera does not see “submarkings for matters inside pages” directly, but the position of finger top of the user relative to the previously identified book page is calculated. To calculate the position of the finger top relative to the book page true way, one need two read-out-able points or symbols with the known distance between them (i.e. the system BO-webcamera—computer have to have a scale). With this aim a book page can comprise two “code-markings for each page” with the known distance between them. Or the every book page can comprise at least two some automatically read-out-able symbols with the known distance between them.

If the a.m. BO-webcamera is fastened to the user, an actual distance between the BO-webcamera and the book page can always change itself a bit, and therefore a constant automatic recalculation of this distance is necessary.

In one other embodiment variant, the system “BO-webcamera-computer” does not calculate the position (coordinates) of the finger top relative to the a.m. book page, but this system identifies a fact of touch of a “submarking for matters inside pages” by a finger top. To avoid an accidental mistaken identification of a “submarking for matters inside pages” because of an accidental touch by a finger of this marking, the system can be adjusted to react only on a double-click of the finger top on the marking (in both above-described variants)

In one other embodiment variant the user does not point by his finger at a marking at all, wherein the “submarking for matters inside pages” are executed as numbers or as letters or as graphical symbols or as any other symbols, wherein after the automatic identification of the actually open page by means of the a.m. “code-marking for each page”, a correspondent to this page menu of numbers (or menu of symbols) appears in a computer display. Wherein the user switches the selected by him numbers (symbols) on, as it is usual for a computer (for example by a finger on a display of a smartphone).

In one embodiment example the positions of two finger tops relative to a book page are observed (watched). If an user slides apart two his finger tops, which are laying on a book page surface (analogically to the case when one executes an enlargement in a display of a smartphone), this part of the book page will be a) presented in a computer display, and b) enlarged.

If a type of a book is so small, that two or more “submarkings for matters inside pages” are covered by a finger top, and therefore one cannot point at a necessary marking in a book page by the finger top, one can firstly provides an enlarged presentation of this peace of text in a computer display (the way as it is described above), and after that one can execute a selection of the markings by the finger top already in a display (through touch of a display surface in a point (area), where the electronic images of the above-described “submarkings for matters inside pages” where appeared).

Analogously but the opposite way one can provide a decreasing. If an user decreases a distance between his two finger tops, which are laying on a book page surface (the same way as one executes a decreasing in a smartphone display), this part of the book page will be a) presented in a computer display, and b) enlarged.

In one embodiment example a position of a finger top relative to a book page is observed, and if one moves a finger top, which one lays on a book page surface, along this page, this part of the book page will be a) presented in a computer display, and b) a picture (an image), or a part of the picture (image), or an image of the whole page, will be changed in the display. It is analogously to the leaf—(turn over)—function in a smartphone, but an user moves his finger top not along a display, but along a book page or along a part of a book page, and this motion results a change of a picture (image) presentation in a display.

As an example, a book can contain a printed picture illustration, with a notice, that besides also other illustrations are supplemented. An user touch the illustration in a book page by his finger top, which touch results the appearing of this illustration in a computer display. The user moves his finger top along the illustration in a book page (analogously as one “leafs”/“turns over” the pictures in a smartphone or Ipad display), and through this motion an illustration in the computer display is substituted by a next illustration etc. In other words, one operates by means of his finger with a printed book page the same way as with a image presentation in a touchable display, and one watches the results of these operations in a computer display.

In one embodiment example an user leads a finger top around some area in a book page (encircles some area in a book page by a finger top), this motion is observed by a BO-webcamera and passed in a computer, then this motion is co-ordinated (compared) with an electronic image of the page, and after that this encircled area appears in the computer display, with the possibility to execute the further usual electronic processing of this electronic image of this area.

Instead of the a.m. BO-webcamera one can use also any other devices, which are able to identify and observe a position or coordinates of a finger top relative to some definite surface. (Like for example a device, which one is placed at a page edge, and which one identifies the motions of a top of a ball-point pen, and then this device presents a written by this pen on this page matter in a computer display. Such devices belong to state of technology).

Instead of a finger top one can use also an auxiliary pointing agent, for example a stick. As this auxiliary pointing agent in particular also a laser pointer can be used. This laser pointer can be fixed in particular on an user's finger. In the case of a laser pointer, after the laser beam has pointed at a selected “submarkings for matters inside pages”, this choice have to be confirmed some way. One can execute this confirmation in particular:
by means of a switcher in a laser pointer; or
through a gesture, which one is recognisable by a BO-webcamera or UO-webcamera, and which one is previously entered in a computer program; or
by voice (an user have to say a code-word).

This way, written briefly & commonly, this interactive book works as follows (s. FIGS. 1-22):

(Among all the embodiment examples of invention only these are illustrated by drawings, because in this case it could be difficult to present an unambiguous description without drawings. Nevertheless all other embodiment examples, which are not illustrated by drawings, are of equal value.)

### BRIEF DESCRIPTION OF THE SEVERAL VIEWS OF THE DRAWINGS

The figures show:

FIG. 1 shows an interactive book in a closed state;

FIG. 2 shows an interactive book in open state: a variant of embodiment example, wherein a BO-webcamera is fastened to user's glasses (spectacles);

FIG. 3-FIG. 6 show an interactive book in open state: a variant of embodiment example, wherein a BO-webcamera is fastened to a book;

FIG. 3 and FIG. 4—a variant of fastening to the book cover;

FIG. 5—a variant of fastening to the book binding textile;

FIG. 6—a variant of fastening to the book bend;

FIG. 7-FIG. 11 show a fastening device to fix a BO-webcamera on a book.

FIG. 7 to FIG. 11 show the succession of steps, how one opens this device from a transportation state and fastens it to a book;

FIG. 12 and FIG. 13 show a fastening device to fix a UO-webcamera on a book;

FIG. 12 shows a fastening device to fix a UO-webcamera on a book separately and independently from the fastening device for BO-webcamera;

FIG. 13 shows a fastening device to fix a UO-webcamera on a book by using the same fastening device for UO-webcamera and BO-webcamera.

FIG. 14 shows how a UO-webcamera can be placed on an upper edge of a book;

FIG. 15 and FIG. 16 show how two or more UO-webcameras can be fastened on book edges;

FIG. 15 shows how two or more UO-webcameras can be fastened on the upper book edges;

FIG. 16 shows how several cameras can be fastened on book edges to give a possibility to compute and determine a current 3D-position of a book relative to a user.

FIG. 17 shows one possible embodiment example of invention, wherein a smartphone or other gadget comprises an additional display, placed on the (in the) glasses/spectacles, or on the (in the) contact lens (or lenses), which display causes an appearing/imaginary/“in the air hanging” image, in particular a book image, which one appears directly (immediate) in front of the user's eyes.

FIG. 18A-FIG. 22 shows an embodiment example, where a display virtual image is created by means of glasses in front of a user similar to a position of a hawker's tray;

FIG. 18A-FIG. 18C shows a position of an appearing/imaginary/“in the air hanging” display image, in particular a book image, relative to a user, wherein a FIG. 18A is a front view, a FIG. 18B is a right side view, and FIG. 18C is a top view.

FIG. 19 shows an electronically adjustable angle between the display image plane and a horizontal plane;

FIG. 20, FIG. 21, FIG. 22 show how a user sees a virtual imaginary display during user's motions.

The a.m. interactive book works, in particular, the following way:

1) One creates a website 1 with the data about some definite books.

This website comprises in particular the following information:
a) to which book corresponds the “general identification code-marking” 2. This marking is coated on (put on) a book 3 (as a paint, lacquer, sticker, etc., in particular also during printing or producing of a book).
b) to which pages 4 of this book correspond the “code-markings for each page” 5. These markings are coated on (put on) each page 4 of the book 3 (also as a paint, lacquer, sticker, etc., also during printing or producing of a book).
c) what information (in particular in a text-, picture-, video-, audio-, etc.—form) corresponds to each of the “submarkings for matters inside pages” 6, which are placed on the each of the above-mentioned identified pages 4. As it is described above, the “submarkings for matters inside pages” 6 can be placed in particular in text blocs 18, in elements inside pictures, etc.

Instead of the website these a.m. data can be placed in particular in an external data carrier (disc, memory-card, etc.), as well as in a data carrier of a computer itself. Among other possibilities a graphical code marking (graphical code) is a regulated collection of paint coatings on a book surface as well as on book pages surfaces. Such code markings are known and they belong to state of technology (as for example, point-codes, dash-codes, etc.).

2) One prints or produces a book 3, comprising the described above “general identification code-marking” 2, “code-markings for each page” 5, and “submarkings for matters inside pages” 6.

3) One connects electronically the BO-webcamera 7 with the computer 8 (through an electric cable 20 or wireless), and one switches on a program for automatic recognising of the a.m. code-markings.

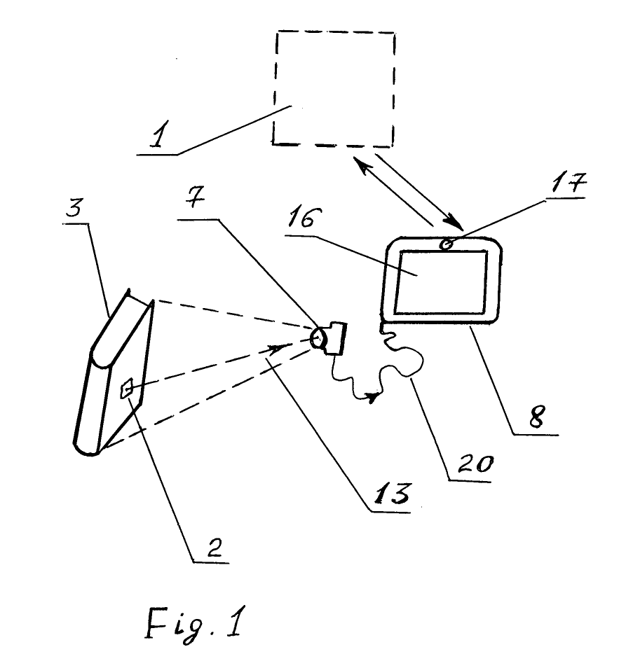

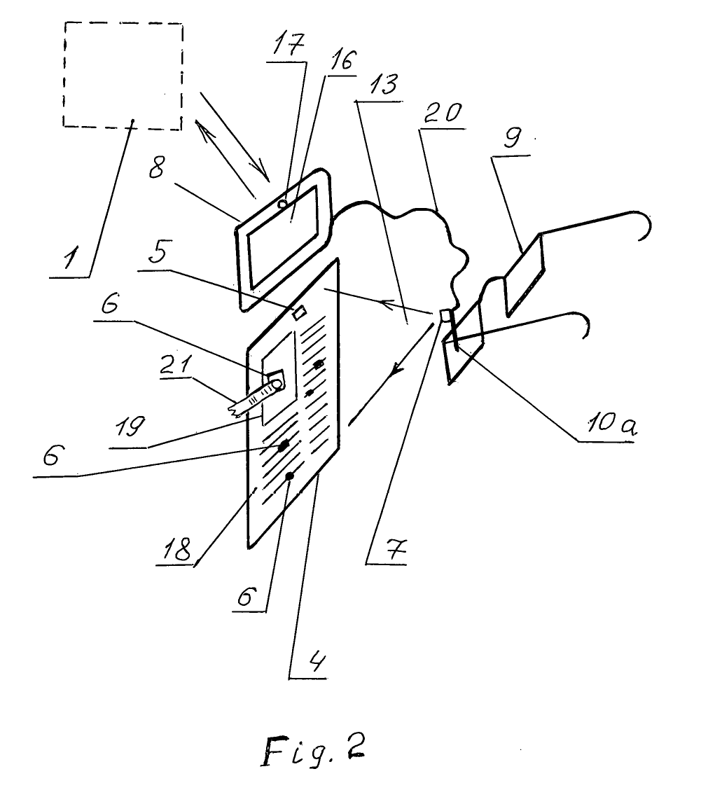

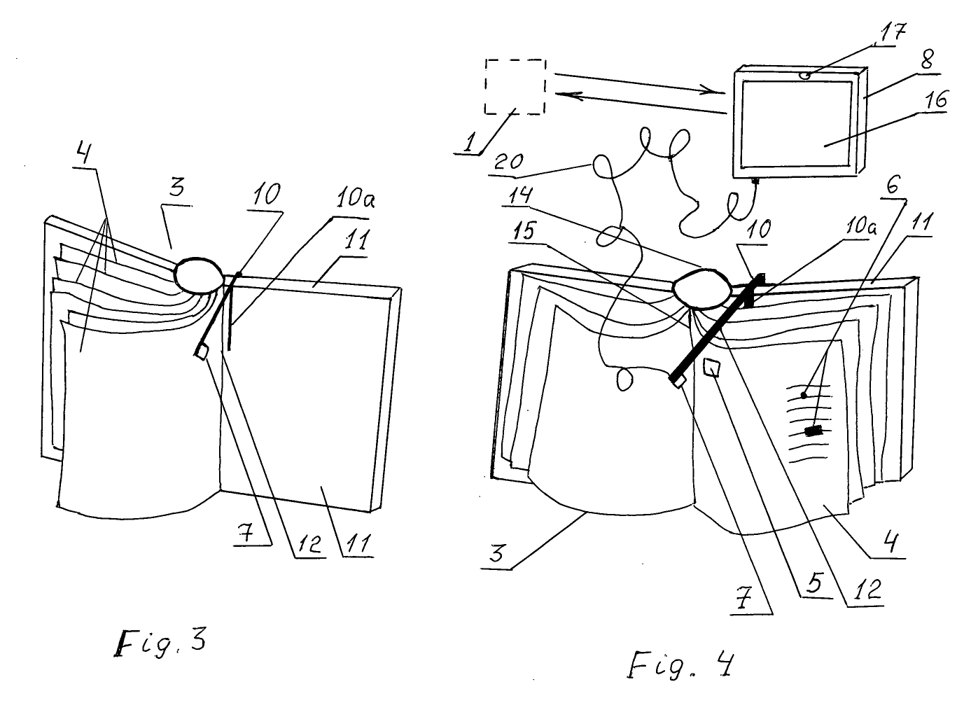

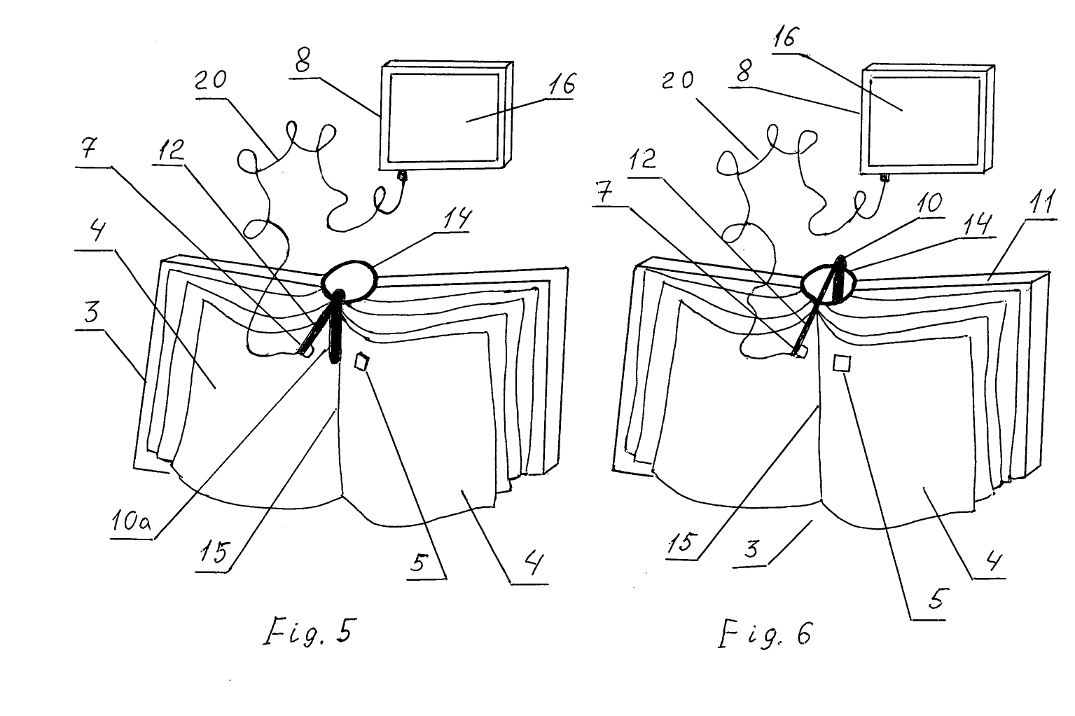

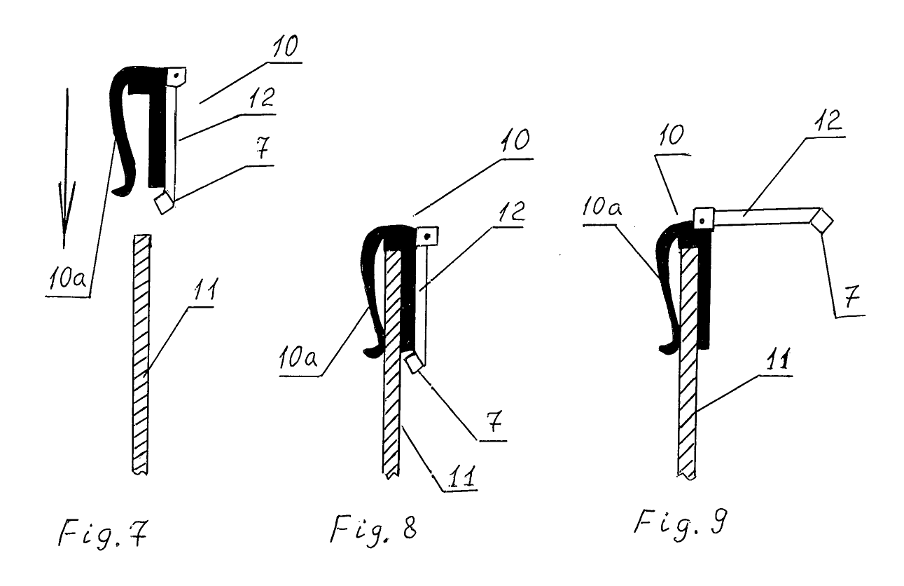

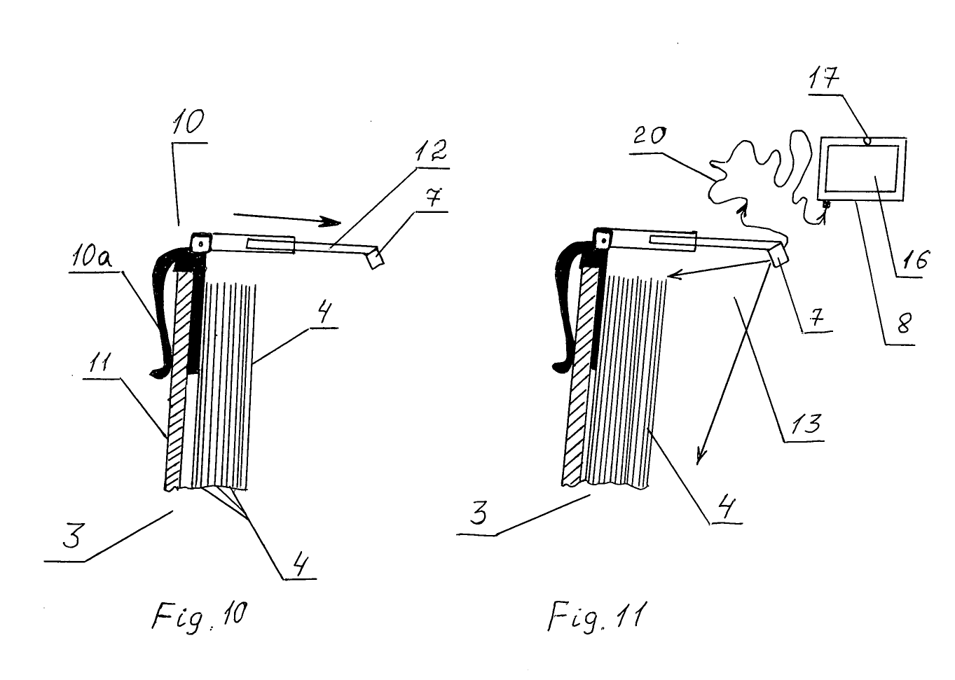

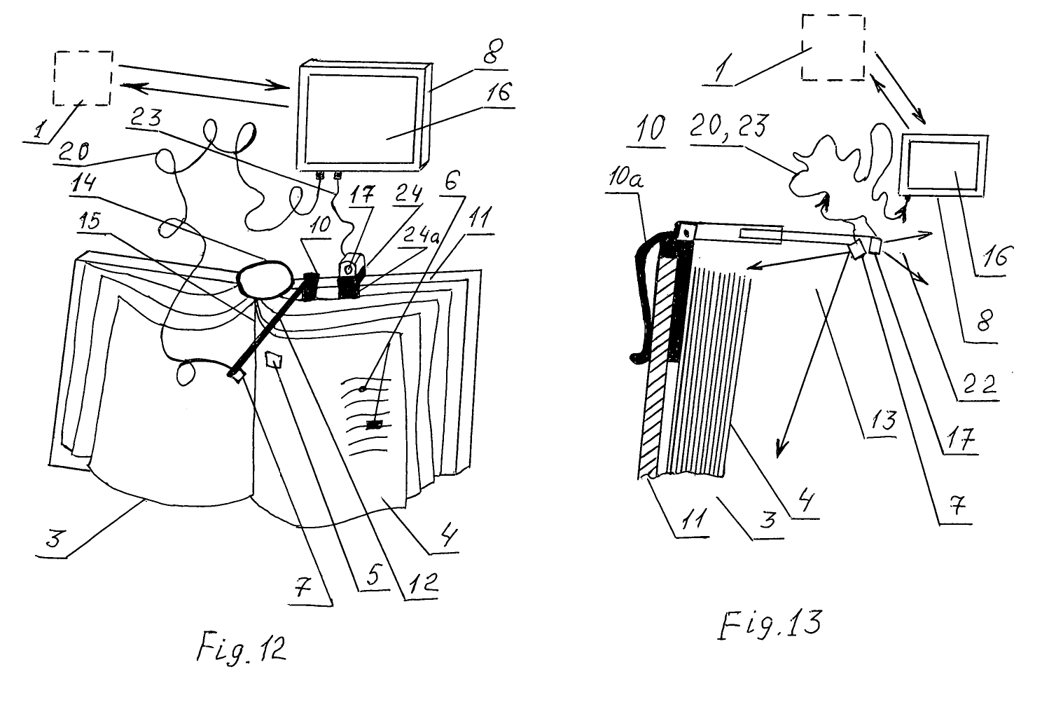

Simultaneously one brings the book 3 in a field of vision 13 of a BO-webcamera 7 (FIG. 1). For this aim one places the BO-webcamera 7 on an user (on his glasses 9 or on his clothes) (FIG. 2), and one directs this BO-webcamera 7 to the book 3 (to the a.m. “general identification code-marking” 2). The BO-webcamera 7 can be fastened not only to an user, but also to a book 3 itself, or it can be placed in an other place. It is only important that the . webcamera 7 have to be directed to the book 3 (to the code markings on this book). If the BO-webcamera 7 is fastened to the book 3, the fastening-device 10 (for example a clamp 10a for a fastening of the BO-webcamera 7, in particular to the back hard cover 11 of the book) comprises a telescopic rod 12, which one is adjustable to a correspondent lengs dependently on an actual book thickness (FIG. 3-FIG. 11). On one end of this rod 12 one places the BO-webcamera 7, and the other end of this rod it fastened to the clamp 10 with the possibility to rotate. This way, the fastening device takes not a lot of space in a transportation mode, but in a working mode one can adjust the rod 12 perpendicular to a book page surface. The BO-webcamera 7 is fastened to the rod 12 such way, that in the working mode a field of vision 13 of the BO-webcamera 7 covers the whole page 4.

The BO-webcamera 7 can be also placed:
on a textile material 14 of the book binding (i.e. on the material, which one connects the hard cover plates),
on a bend 15 of a book cover, or
not only on the a.m. back plate of a book cover, but also on a front plate of a book cover,
or similar.

4) Computer 8 (or a device for processing of information from the BO-webcamera 7) reads the “general identification code-marking” 2, connects itself with the website 1, and identifies the book 3 by means of the “general identification code-marking” 2 of this book according to data from the website 1. After that the computer 8 informs the user about readiness to the next step—following identification of pages. If necessary, the computer 8 presents in it's display 16 an information, which one corresponds to this book in general (still without consideration of the pages).

5) The user opens the book 3 on some definite (no matter what one) page 4. This way the “code-marking for each page” 5, which one corresponds to this page 4, will be introduced (brought in) in the field of vision 13 of the BO-webcamera 7 (FIG. 2 and FIG. 3-FIG. 6). Computer 8 (or a device for processing of information from the BO-webcamera 7) reads an actually opened “code-marking for each page” 5, connects itself with the website 1, and identifies the opened page 4. After that the computer 8 informs the user about readiness to the next step—following identification of submarkings for matters inside the page. If necessary, the computer 8 presents in it's display 16 an information, which one corresponds to this actually opened page in general (still without consideration of the submarkings 6 for matters inside the page 4).

6) The user shows (points) by his finger 21 at one of the “submarkings for matters inside pages” 6 inside the actually opened page 4 (in particular with a double-click to avoid mistaken identifications). Computer 8 with the BO-webcamera 7 recognises a position of the finger relative to an actually pointed marking 6, and represents the correspondent datas in the display 16 (all further or related steps are already described above).

7) Or instead of the previous item (6) (or additionally to the previous item (6)) the computer 8 with the UO-webcamera 17 recognises a position of user's eyes pupils, or of the user's face muscles changes of state due to facial expressions, and dependently on these eyes pupils positions or face muscles state changes the computer represents the correspondent datas in a display 16 (also see above for details).

To recognise by means of computer 8 the positions or the changes of positions of eyes or eyes pupils (pupillen) relative to a book page 4, the user can firstly direct his eyes pupils (his look) at least at two points or other markings, wherein these points (markings) were put on the book page before, and wherein the positions of these points (markings) relative to other represented in a book page 4 visual information is known to the computer 8. This way the position of eyes pupils relative to the book page 4 (i.e. where the user looks at) is adjusted.

The system “computer 8—UO-webcamera 17” can execute this adjustment also automatically (in particular also it can be executed always repeatedly many times), during the reading of word sentences in the page 4 by the user. The system “computer 8—UO-webcamera 17” can find a correlation between the positions of first and last word of the first sentence (as well as also for further sentences) in the page with the extreme positions of eyes pupils by reciprocating motion of eyes pupils during a reading. The UO-webcamera 17 is directed in the opposite direction relative to the BO-webcamera 7, i.e. the UO-webcamera 17 has the user (in particular his face or eyes pupils) in the UO-webcamera field of vision 22. The UO-webcamera 17 is connected with the computer 8 by means of a cable 23 or wireless.

The UO-webcamera can be fastened on the computer, on a table, on a book, etc. A fastening device 24 (for example a clamp 24a) for a placed on a book UO-webcamera can be executed separately & independently from the fastening device 10 for the BO-webcamera (FIG. 12), or both UO-webcamera 17 and BO-webcamera 7 can be fastened on the same fastening device 10 (FIG. 13).

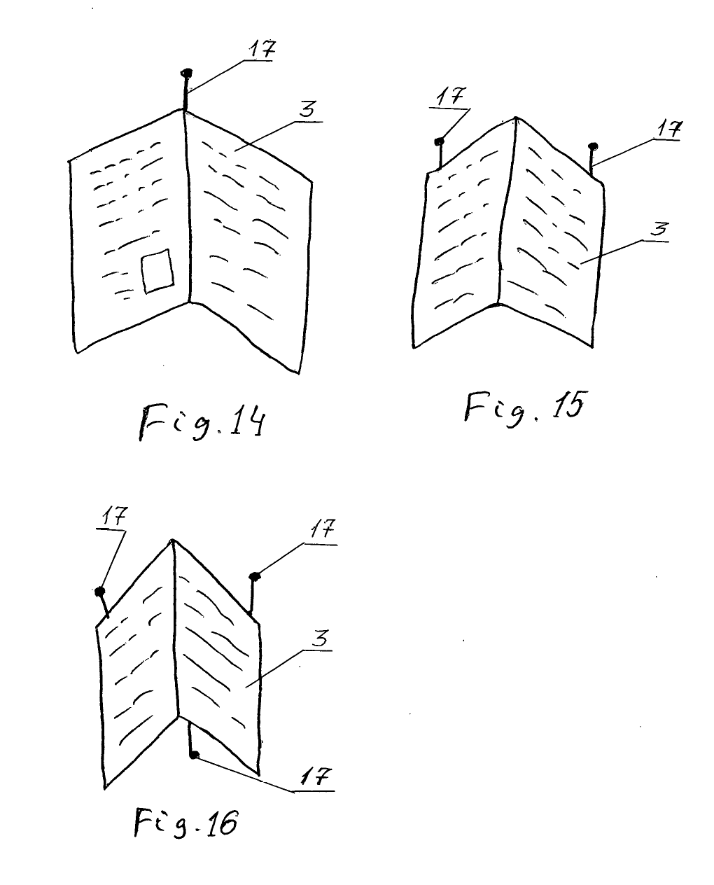

The UO-webcamera 17 can be also placed on an upper edge of the book, approximately in it's middle (FIG. 14). Also two or more UO-webcameras 17 can be fastened on the edges, in particular on the upper edges (FIG. 15, FIG. 16). Using of several cameras with the correspondent electronical processing device, in particular also with the possibility to get informations from the BO-webcameras, gives a possibility to compute and determine the current 3D-position of a book (or of the open book pages) relative to the user or to his eyes pupils.

Instead of the paper pages of a book one can use also a display of a e-book (kindle) or a display of a computer. I.e. one can operate with an information, which one is represented optically in a display of an electronic device, without a direct electronic connection with this device.

In one embodiment example the above-mentioned operations can be executed by the computer 8 only, wherein one downloads from a website 1 (or from an external data-carrier) only data or software. In one other embodiment example the above-mentioned operations can be executed by means of a website only (i.e. by means of devices (server), which provide the website functioning), wherein the system “computer—webcamera” delivers in the website only the data, and after that these data are processed there in the website, and the results of this processing are delivered back to the computer. Between these extreme variants a whole spectrum of embodiment examples can be executed, wherein the delivered by webcamera data are processed partially by the computer 8 and partially by the website 1.

This way, in an embodiment example, which one concerns an interactive book, the interactive (intercommunication-able) book comprises the following main elements:
a book;
“general identification code-marking”.
“code-markings for each page”;
“submarkings for matters inside pages”;
computer;
BO-webcamera or UO-webcamera or both;
if necessary, other connected with the computer devices for observation of reactions of the user (a.o. of his eyes pupils position relative to a book page, facial expressions characteristics & changes, gesticulation, medical-biological parameters and reaction and the similar);
website,
   wherein
definitions of these elements (i.e. what is meant under these elements),
detailed description of these elements,
description how these elements are connected one with another, and
interaction of these elements
   are described above.

In one embodiment-example the computer comprises one or several UO-webcameras or also other kinds of usual peripherals for interactive communication of an user with the computer or with a computer game, wherein facial expression (state of facial muscles) or the facial expression changes (tensions of the facial muscles) of the user, among other possibilities also muscles tensions of the eyes—and eyes pupils of the user, are watched by a computer through a UO-webcamera or UO-webcameras, this information is passed to the computer, also this information is converted in a digital (electronic) form, analysed and processed, and after that the computer changes the displayed matter in it's display according to this information, or the computer changes the current reactions of a computer game on the user's actions.

In one embodiment-example the computer watches through the UO-webcamera the position of the eyes, passes this information in computer, after that this information is converted in the digital (electronic) form and processed, after that the computer changes the picture (displayed matter) on the display correspondently to this information, wherein simultaneously
a) the dimensions of the pupils or changes of the pupil's dimensions and
b) the position of the eyes or of the pupils relative to (with respect to) the display (u.a. relative to the virtual display, which one appears through display-spectacles)
   are watched by the UO-webcamera and processed by the computer, and on the grounds of the results of this processing the correspondent part (segment) in the picture in the display (in the displayed matter) is enlarged or decreased.

In one embodiment-example the user directs his eyes or pupils (i.c. looks) at one definite point in the display, and he screws his eyes up or blinks by his eyes, the UO-webcamera and computer recognise these facial expression changes in the muscular system of the face, and the computer enlarges in the display this corresponding surface area of the displayed matter, at which surface area the user looks, or the computer stops the enlarging when this above mentioned eye screw-up muscle relaxes, or the displayed picture is decreased if one executes some determined facial expressions (face muscles states) on his face (in particular, one relaxes the eye muscles (one makes larger his eyes), one opens his mouth, or one uses any kind of other facial expression-forming movements of the face muscles, wherein a correspondence between the said facial expression-forming movements of the face muscles (or face muscle tensions) and the reactions of the computer can be adjusted beforehand; or an user can direct the eyes (look) at a displayed on a display enlargement-decreasing scale (line), then to focus the pupils of the eyes on a definite point of this scale, then to screw the eyes up or to blink by the eyes, and this way the computer recognizes through the UO-webcamera the preferred by an user resolution (grade of enlargement or decreasing), and the computer adjusts (set) this grade of enlargement or decreasing in a display.

In one embodiment-example the enlargement/decreasing scale (line) appears in a display, an user directs the eyes (or pupils of the eyes) at a definite point of this scale, the user focuses the eyes (makes smaller the pupils of the eyes), or the user screws his eyes up or blinks by his eyes, and this way the computer recognizes through the UO-webcamera the preferred by an user resolution (grade of enlargement or decreasing), and the computer adjusted (set) this grade of enlargement or decreasing in a display, wherein also a marker (as for example a little quadrangle) can appear on the that point of the above mentioned scale, which point is selected by the user the above described way.

In one embodiment example the computer game (a system) comprises a lie-detector, wherein the computer game asks the user questions (verbally, i.e. with the words, or by proposing of different choices of verbal or motorical, or both, reactions or situations in a game), and, dependently on his answer (or dependently on his above mentioned choice of reaction or situation in the game), in connection with the processed by the computer readings/indications of the lie-detector, the computer game choose a variant of the further behaviour (situation, reaction, program, velocity of actions, etc.) of the computer game with the user.

In one embodiment-example, instead of—(or additionally to) a lie-detector, the computer game (the system) comprises an encephalograph, a myograph, or any other medical-diagnostic equipment, which equipment executes the reading of the current medical-biological parameters (in particular finally of the emotional state) of an user, watches these parameters and passes these data into the computer, after that these data are processed by a program of the computer game, and, dependently on the results of this processing, the computer game choose a variant of a further, either current in the moment or strategical, behaviour with the user (situation, reaction, program, velocity of actions, variants of the verbal (with the words) answers or communications, etc.).

In one embodiment example, which one comprises a creating/forming of a visual information in a display of a smartphone or mobile telephone, wherein these signals, which are normally causing a real image on a display of a smartphone/mobile telephone, are converted in an image, in particular in a virtual image (appearing/imaginary/“in the air hanging” image), which one appears directly (immediate) in front of the user's eyes, in particular this displayed matter (image) is created/formed in the lenses of glasses (spectacles), or in the lenses cover pieces, or only in one of the two lenses cover pieces, or in the eyes contact lenses, or only in one of the two eyes contact lenses.

In one embodiment example a smartphone or a mobil telephone comprises an additional display, which display is placed on the (in the) glasses/spectacles, or on the (in the) contact lens (or lenses), and besides, in particular, this display is connected with a mobile telephone device electrically or electromagnetically by a wire or wireless, wherein the signals, which are normally causing a real image on a display of a smartphone/mobile telephone, are converted in an image, in particular in a virtual image (appearing/imaginary/“in the air hanging” image), which one appears directly (immediate) in front of the user's eyes, in particular this displayed matter (image) is created/formed in the lenses of glasses (spectacles), or in the lenses cover pieces, or only in one of the two lenses cover pieces, or in the eyes contact lenses, or only in one of the two eyes contact lenses.

In one embodiment example a smartphone or a mobil telephone comprises two displays, each one for one of the eyes, and also it comprises a.o a hardware and software for a creating of 3-D images or for a presenting of stereopictures.

In one embodiment-example the computer watches through the UO-webcamera the position of the eyes, passes this information in computer, after that this information is converted in the digital (electronic) form and processed, after that the computer changes the picture (displayed matter) on the display correspondently to this information, wherein simultaneously
a) the dimensions of the pupils or changes of the pupil's dimensions and
b) the position of the eyes or of the pupils relative to (with respect to) a book page (or relative to a page of a e-book/kindle)
   are watched by the UO-webcamera and processed by the computer, and on the grounds of the results of this processing the correspondent part (segment) in the picture in the display (in the displayed matter) is enlarged or decreased.

In one embodiment example an user directs the eyes or pupils of the eyes at (looks at) one definite point in a book page and screws his eyes up or blinks by his eyes, the UO-webcamera and computer recognise these facial expression changes in the muscular system of the face and present a correspondent picture (displayed matter) in a display, and then
the computer enlarges in the display this corresponding surface area of the displayed matter, at which surface area the user looks, or
the computer stops the enlarging when this above mentioned eye screw-up muscles relaxes, or
the displayed picture is decreased if one executes some determined facial expressions (face muscles states) by his face (in particular, one relaxes the eye muscles (one makes larger his eyes), one opens his mouth, or one uses any kind of other facial expression-forming movements of the face muscles), wherein a correspondence between the said facial expression-forming movements of the face muscles (or face muscle tensions) and the reactions of the computer can be adjusted beforehand;
or an user can direct the eyes (look) at an enlargement-decreasing scale (line), which one is placed in a book page or in a display, then focus the pupils of the eyes on a definite point of this scale, screw the eyes up or to blink by the eyes, and this way the computer recognizes through the UO-webcamera the preferred by an user resolution (grade of enlargement or decreasing), and the computer adjusts (set) this grade of enlargement or decreasing in a display.

In one embodiment-example the enlargement/decreasing scale (line) appears in a display, an user directs the eyes (or pupils of the eyes) at a definite point of this scale, the user focuses the eyes (makes smaller the pupils of the eyes), or the user screws his eyes up or blinks by his eyes, and this way the computer recognizes through the UO-webcamera the preferred by an user resolution (grade of enlargement or decreasing), and the computer adjusted (set) this grade of enlargement or decreasing in a display, wherein also a marker (as for example a little quadrangle) can appear on the that point of the above mentioned scale, which point is selected by the user the above described way.

In one embodiment example the claimed device uses an e-book (kindle) instead of a book, wherein the device comprises means for current transfer of information in the computer, which means transfer in the computer the information about an actual page and about a current eyes pupils position relative to this page as well as about a current facial expression. Wherein the information about the actual page is transferred from the e-book/kindle, and the information about the current eyes pupils position and current facial expression is transferred from the UO-webcamera.

In one embodiment example one uses an e-book (kindle) instead of a book, wherein an information about a current page and about a current eyes pupils position relative to this page as well as about a current facial expression is currently transferred in the computer. Wherein the information about the actual page is transferred from the e-book/kindle, and the information about the current eyes pupils position and current facial expression is transferred from the UO-webcamera.

An aim of the presented in the claims invention is also to provide a method, by which one could see, use and process interactively a big interactive image or big interactive keyboard, without a necessity to carry a big physical device. It concerns both a) operations with virtual books, and b) operations with a virtual (imaginary, “in the air hanging”) display of an electronic device in general, independently of the subject content of a displayed image (i.e. no matter whatever one sees in this display). Therefore the all below described matters concern both a) method and devices for presentation and operations with virtual books and b) methods and devices for presentation and operations with any kind of information (text, pictures, any kind of a smartphone display content, etc.), which information is presented in a virtual (imaginary, “in the air hanging”) display of an electronic device.

The attained by this inventions advantages are, in particular, that it makes it possible to provide a possibility to operate with a large imaginary interactive image, as well as with a large imaginary interactive keyboard, independently on the dimensions of a physical electronic device, which one provides these above-mentioned operations.

Definition: In further description below under the term “Gadget” one understands an I-Phone or any other smartphone, mobile telephone, I-Pad, I-Pod, any kind of a tafel-PC, also a laptop, any kind of PC, and any kind of electronic device, which one is able to execute functions of a personal computer.

One advantageable further development of the invention is described below. This further development makes it possible to provide a secure using of a smartphone (or any other gadget) only by an authorised for it person.

One next advantageable further development of the invention is further described below. This further development makes it possible to provide different improved executions of 3D-images and stereacoustic representations.

Some embodiment examples of the invention are described below.

In one embodiment example of the method a visual information is presented in an interactive touchable (touch-sensitive) display of a gadget, and this visual information is operated by a finger (this finger will be further named below as a “working finger”), or this visual information is operated by a mediator (middle-body), for example by a stick or needle (further named below as a “middle-stick”), for which purpose one touch by the a.m. working finger or by the a.m. middle-stick a correspondent virtual picture (a.o. an icon—i.e. a graphic symbol in a display) or virtual keys in this display. Furthermore, this visual information is simultaneously presented immediately (directly) in front of the eyes as an imaginary image (virtual, aerial image, picture outside of a screen surface), for which aim this a.m. visual information is transferred from the gadget in a looking device (monitor), which looking device is placed in glasses (spectacles) or in glasses extension, or in an other extension piece placed on a user's head, or in contact lenses (this looking device is further named below as “glasses looking device”).

And simultaneously an every position (spatial coordinates or 2D-coordinates in a display surface) of the a.m. working finger (or of the a.m. middle-stick, or only of their parts or only of tops or end points of the a.m. working finger or middle-stick) is located by a special device, and this information about this position is transferred in the glasses looking device in the real-time regime,

wherein a virtual image of this working finger or of this middle-stick is superimposed on a picture (image), which one is transferred from the gadget display in the glasses looking device (i.e. in other words, the two above-mentioned images (the first one—from the gadget display, and the second one—from the working finger (or middle-stick) position relative to the gadget display), are superimposed or put over each other in the same picture (image) in the glasses looking device.

Here instead of a working finger (or of a middle-stick) one can present in a glasses looking device only a virtual image of it's top (or end point), a.o. in a form of a cursor (as for example cross or arrow), wherein one can locate only two coordinates of the working finger top (or of a middle-stick top), in the plane of gadget display surface, without taking into account of a vertical distance between the working finger (or middle-stick) and gadget display surface.

Besides, a.o., when executing of “enlargement” and “decreasing”—functions also an image of a second finger (or it's top) can temporary appear in a glasses looking device, after one has touched the gadget display by this finger, together with a working finger, wherein a.o. a forefinger can be used as a working finger, and a thumb can be used as a second finger. Here a top of the working finger (or of the middle-stick) can be marked with any marker (a.o. magnetic marker, electrostatic marker, optic marker, etc.), to indicate a position of the a.m. top, wherein instead of a single finger one can indicate several, up to 10 working fingers, locate their spatial positions, and use them to operate the system.

Besides, one can a.o. generate the a.m. imaginary image only in front of one of the two eyes, for which aim a.o. one can transfer a visual information e.g. in a glasses extension, which one is placed on one from the two glasses lenses.

And besides, the a.m. imaginary picture (image) a.o. can occupy either a whole field of vision or only a part of the whole field of vision, wherein a rest part of the field of vision remains for the surroundings observation; or a field of vision can be occupied by several imaginary pictures (images), which pictures (images) a.o. also can be transferred in the glasses looking device from several sources (a.o. from several gadgets).

This method can be also executed such way, that a visual information is not presented in the physical touch-sensitive gadget display, and the visual information is not operated by touching of the physical display, and instead of it an every position (spatial coordinates or 2D-coordinates in some plane) of a working finger (or of several fingers, or of a middle-stick, or only of their parts, or of tops (end points) of the a.m. working finger or fingers or middle-stick) is located by means of a special device, and this information about this position is transferred in the glasses looking device in the real-time regime,

This way a virtual image of this working finger or of these fingers or of this middle-stick) is superimposed on a picture (image), which one is transferred from the gadget in the glasses looking device (i.e. in other words, the two above-mentioned images (the first one—from the gadget, and the second one—from the working finger—(or middle-stick)—spatial position), are superimposed or put over each other in the same picture (image) in the glasses looking device, and this way the whole system is operated by the finger-controlled operations with the virtual (imagionary/appearing, aerial image-like, “in the air hanging”) interactive image.

In one embodiment example of the method a virtual imaginary interactive image of a music keyboard is formed in the glasses looking device, which way one can play by means of the virtual claviatur the same way as one plays by means of a real keyboard claviatur, a.o. by 10 fingers.

In one embodiment example of the method a virtual imaginary interactive image of a PC-keyboard is formed in the glasses looking device, which way one can operate by means of the virtual claviatur the same way as one operates by means of a real PC-keyboard claviatur, a.o. by 10 fingers.

In one embodiment example of the method the every position (spatial coordinates or 2D-coordinates in the display surface plane) of the a.m. working fingers (or of a single working finger, or of the a.m. middle-stick, or only of their parts or only of tops (end points) of the a.m. working fingers or middle-sticks) is located by the special device, and after that this information about this position is transferred in the a.m. glasses looking device in the real-time regime), wherein this special device is placed in the glasses looking device or in any kind of a head cover, wherein the a.m. special device can be placed a.o. also on a wrist, or on a waist belt, or on any other part of the body, or on a table, or on any other kind of support or carrier near the working finger.

In one embodiment example of the method the visual information is not presented in the physical touch-sensitive gadget display, and the visual information is not operated by touching of the physical display, and instead
the whole system is operated through a touchpad (a.o. the same way as it takes place in a laptop).

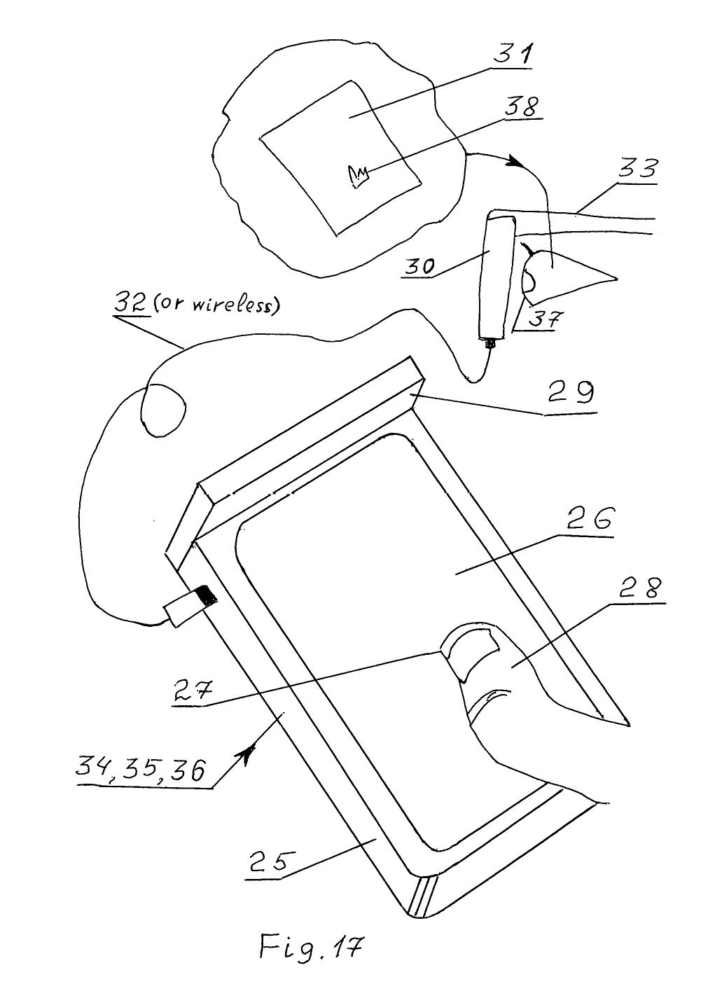

One possible embodiment example of invention is presented in FIG. 17. In this example of embodiment the device comprises:
a smartphone (or I-pad, or an other gadget) 25 with an interactive display 26, which one can be controlled usual way through touching by a top 27 of a finger 28 or by two tops of two fingers;
a device 29 for identification of a location of a finger top relative to display 26 (i.e. for identification of a point in a display 26, above which point (without obligatory contact with this point) a top of a finger or a top of a middle-stick is currently located); wherein this device 29 (further named as a “location searching device”) can be installed in (mounted on) a smartphone, I-pad or any Gadget 1 both permanently as a gadget's part and also it can be executed as a removable device;
a device 30, by means of which one a visual information, which one appears in a display 26, is simultaneously presented immediately (directly) in front of the eyes 37 as an imaginary image (virtual, aerial image, picture outside of a screen surface) 31, for which aim this a.m. visual information is transferred from the device 25 in a device (looking device) 30 through a cable 32 or wireless; which looking device 30 is placed in glasses (spectacles) 33, or in glasses extension, or in an other extension piece placed on a user's head, or in contact lenses directly on the eyes (this looking device is named here as “glasses looking device” 30), wherein this “glasses looking device” 30 can be provided with two separate electric inputs with the aim to present different video informations in front of the left and right eyes, and this way to provide a 3D-visual picture presentation;
an electronic device 34, which one converts (represents) the location of the finger top 27 relative to display 26 in electronic form and then represents (visualises) this location inside the a.m. visual imaginary image 31 as a finger top image 38 or as a cursor 38 such way, that the location of this finger top image or of this cursor corresponds exactly to a point in the image, above which point (above the display 26) this finger top or middle-stick top is physically located.

(The functions of this a.m. electronic device 34 can be executed, completely or partially, both by an additional chip, and by a main processor of the device 25);
an electronic device 35, which one converts (represents) the coordinates of a top of a finger or of a middle-stick (relative to interactive display 26) in an electronic form, and then this device 35 enlarges a part of an image, which one is located in the display surface immediately under the finger top or middle-stick top, wherein this local image enlargement can be represented also by a glasses looking device 30 in an image 31 under the cursor 38. (The functions of this a.m. electronic device 35 can be executed, completely or partially, both by an additional chip, and by a main processor of the device 25);
an electronic device 36, which one sends the different visual informations to the left and right parts of the glasses looking device 30, to provide a 3D-image in front of the eyes. (The functions of this a.m. electronic device 36 can be executed, completely or partially, both by an additional chip, and by a main processor of the device 25);
a connecting cable 32, which one connects electrically the gadget 25 with the glasses looking device 30, which way, through the transfer of electric signals, the a.m. image 31 appears in the glasses looking device 30 simultaneously with the appearing of the same or similar image in the display 26, together with the image of a cursor 38, which one shows the location of a finger top 27 (or of a middle-stick top 27) relative to the display 26.

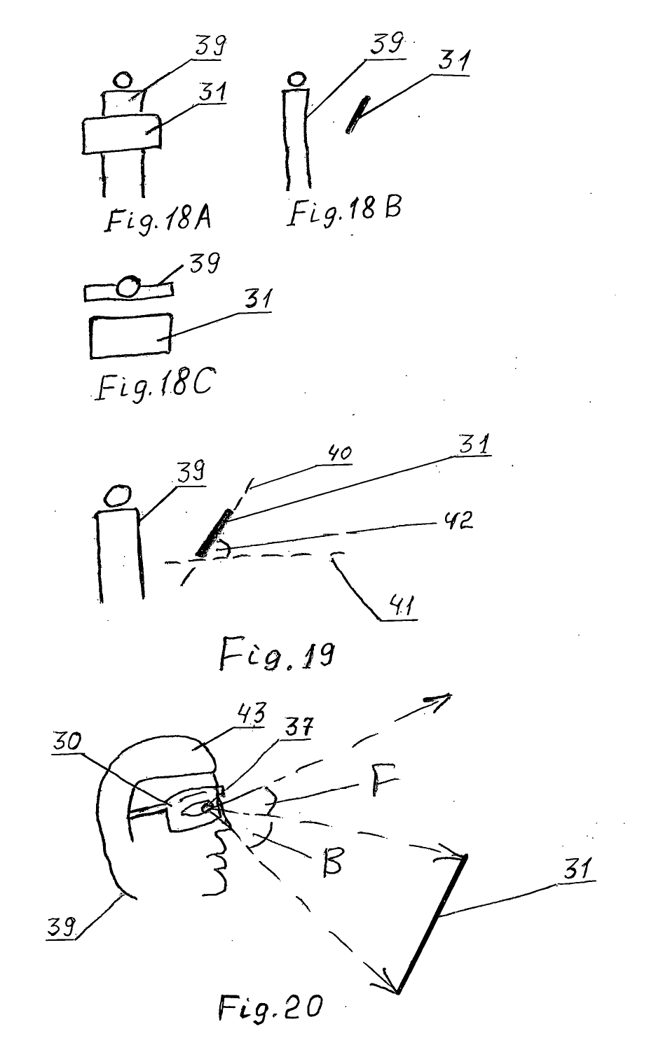

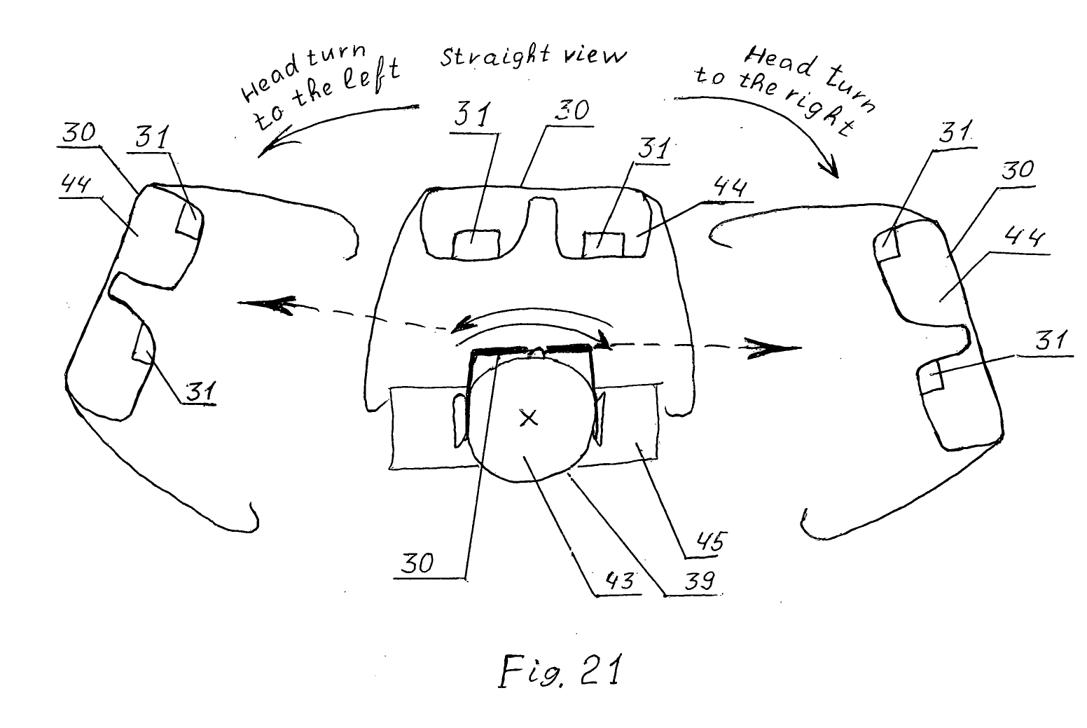

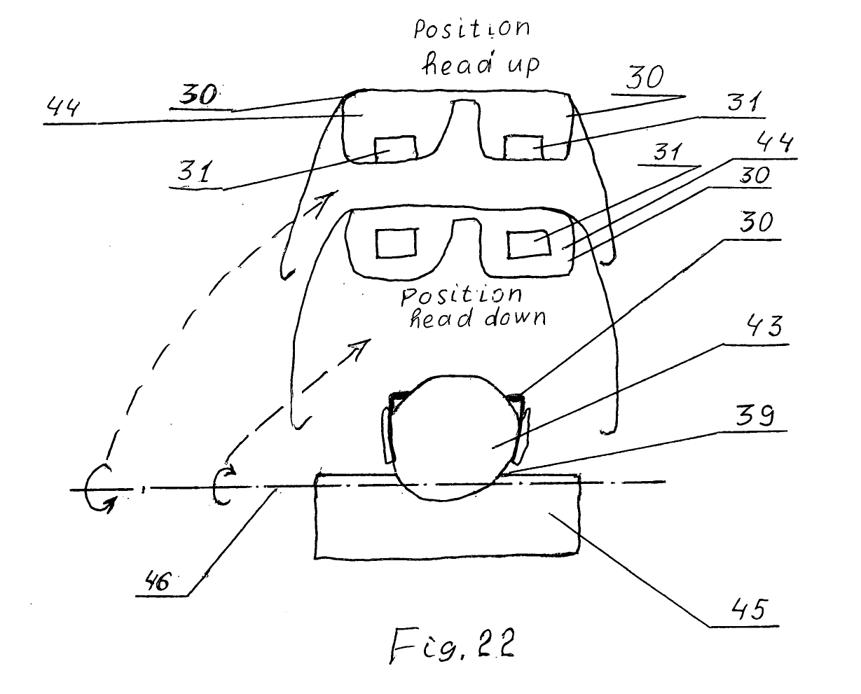

In one other embodiment example a display virtual image 31 is created by means of glasses 30 in front of a user 39 similar to a position of a hawker's tray (FIG. 18(A-C), FIG. 19, FIG. 20, FIG. 21, FIG. 22, wherein the FIG. 18A is a front view, FIG. 18B is a right side view, FIG. 18C is a top view). (In all cases this virtual image is created by means of the correspondent glasses 30 or by means of eyes contact lenses.) Here the position of this a.m. display image 31 relative to the user 39 differs from the position of this hawker's tray only in angle to horizontality: plane of this display image 31 is not horizontal, but lays in a plane 40, which one forms an angle 42 with the horizontal plane 41 (FIG. 19). This angle 42 can be also electronically adjustable, and it's value can vary from 0 degrees to 90 degrees. As well as a distance between this display an user, and an exact position of this display relative to user can be adjustable dependently on the user's settings. Practically it can be executed by moving of fingers in front of the eyes, if the correspondent settings are made. The necessary for it electronic technique belongs to state of technology.

In other respects, during the user's motions as well as during the motions of the user's parts of body (head 43 , eyes 37 , etc.) an user 39 sees a position of this virtual display 31 the same way, without difference, as a tray hawker sees the top surface of his tray. I.e. when the user bends his head 43 down, or the user looks with his eyes down, wherein his look is located inside a field of vision “B” (or “Display”), he sees the display image (FIG. 20, FIG. 22). When lifts the user his head or eyes or both up, wherein his look is located inside a field of vision “F” (or “Far”), he sees the surroundings (for example street, buildings, people) in front of him.

In this moment the display image 31 remains below, in the lower periphery of the user's view (FIG. 20, FIG. 21, FIG. 22). Or the user does not see this display at all, if such an electronic setting was made (i.e. if it was electronically adjusted, that the device, which generates the display image, swithes off this display image, if the user does not directs his eyes to this display).

If the user turns his head or his eyes on the right (relative to his body or relative to his shoulder line 46), he sees surroundings to his right. In this moment the display image 31 remains to the left, in the left periphery of the user's view, or he does not see this display at all.

If the user turns his head or his eyes on the left (relative to his body or relative to his shoulder line 46), he sees surroundings to his left. In this moment the display image 31 remains to the right, in the right periphery of the user's view, or he does not see this display at all, if such a setting was previously electronically executed (s. above).

If user looks straight (forward) or he lifts his eyes up, he can turn his head or his eyes horizontally on 180 degrees from the left to the right and vice-versa, and this way he can observe the surroundings, wherein the display image does not interfere.

If user looks at the display image, all of the surroundings (both nearly and far located) remain on a periphery of his view, but not completely outside the view of his attention, the same way as it takes place by operations with a small device (gadget).

If an user 39 turns his body completely (i.e. body and head together), he sees that the virtual display 31 turns itself with the user together, the same way as it takes place in the case of a hawker's tray. In further description the above-described virtual display 31 is called as a “virtual tray—display”.

In one other embodiment example of invention no observation of eyes pupils positions takes place, i.e. the system does not contain cameras or sensors or any other means for watching of the eyes pupils positions of an user. But the system observes a position of user's head. I.e. the system watches how the user turns his head to the left and to the right relative to a vertical axis; and how the user bends his head down and lifts his head up relative to an axis, which one passes through the left and right shoulders 45 and neck. Or also, if necessary, how the user shakes his head. In other respects, all given above descriptions are completely valid also for the case, when no observation of the eyes pupils positions takes place, i.e. for the case, when the system does not comprise cameras or sensors or any other means for observation of positions of the user's eyes pupils. But in this case one operates with the display by fingers or by voice, because the operations with the eyes pupils position changes is impossible.

It is important, that:
the window with a content of the display (i.e. the above-described virtual image) occupies only a part of a whole field of vision, and also that
from the user's point of view this window remains always in the same position relative to the user's body, when the user moves his head and his eyes.

This way the user can always observe the real surroundings 44, and he can many times change the direction of his attention from this surroundings 44 to the above-mentioned display window (display 31) and back to the surroundings again. The real surroundings 44 can be seen by the user 39 through the transparent material (lenses of spectacles) in the glasses (spectacles) 30 directly. Or also, as an other embodiment example, these surroundings can be observed by a video camera, and then represented by a computer in a display in front of the user's eyes. Wherein the user does not see these surroundings directly, but he sees these surroundings in a display. Wherein this display occupies all field of vision of this user. Nevertheless, evidently, this variante is less comfortable.

The above-described embodiment examples are schematically presented in the drawings FIG. 18(A-C), FIG. 19, FIG. 20, FIG. 21, FIG. 22.

In the opposite, in one other embodiment example the whole user's field of vision is occupied by the virtual display (as it takes place in computer games, where one uses glasses (spectacles) or helmets. This way an user basically cannot move himself safe, free and far, because he sees only a virtual reality, but not real surroundings. In particular one cannot read a virtual book and observe surroundings simultaneously, what is usual for book reading in public places as for example in a transport or on a street bench. To solve the problem of a free motion, the computer have to observe and analyze the real surroundings and to install them in the virtual reality. In this case the placed on an user videocamera fixes also surroundings (landscape, obstacles, room—or space dimensions, all things in a room or in an environment), and all kinds of obstacles at all. Then these circumstances are transferred into computer, after that the computer processes this information and integrates these a.m. obstacles in a virtual reality, which virtual reality is then shown to the user through the glasses looking device. Furthermore, these things and obstacles can be integrated in the virtual reality either in an optically not-changed form, or they can be optically transformed in some other things, obstacles, persons, animals, etc., which objects, nevertheless, have the same dimensions and, if necessary also the same parameters of motion. For example, an user could see a modern computer table as an animal, which one has the same dimensions, and therefore the user have to go round this piece of space when the user moves himself, regardless of what the user sees in this piece of space.

In this embodiment example one can also use computer games in a regime of a not-limited movement for user, in particular, for example, one can run, ride a bicycle or drive a car real way, wherein, nevertheless, the user does not see the real surroundings. Instead, the user sees virtual surroundings, wherein the real objects and obstacles (inclusively in particular roadway contours), are installed (integrated) in a virtual reality either without changes, or in some changed form. For example, if user runs through a forest, and he has put on an above-mentioned glasses looking device, wherein this glasses looking device blocks a whole real view, he can see nevertheless the walking way with all obstacles and trees. But instead of obstacles in a form of stumps he could see for example dragons, which have the same size and dimensions, as well as, for example, he can see fantastic animals on trees branches. If one drives a car, he can see, for example, a fantastic landscape instead of a real roadway contour of an autodrom (motor-racing circuit), wherein, nevertheless, these fantastic landscape and real roadway contour of an autodrom (motor-racing circuit) have the same road contours.

In one other embodiment example, wherein a visual information is presented in a gadget display, a.o. in an interactive touchable (touch-sensitive) gadget display (a.o. of a smartphone or of an Ipad etc.), and this visual information is operated by a finger (this finger will be further named below as a “working finger”), or this visual information is operated by a mediator (middle-body), for example by a stick or needle (further named here as a “middle-stick”), for which purpose one touch by the a.m. working finger or by the a.m. middle-stick a correspondent virtual picture (a.o. an icon—i.e. a graphic symbol in a display) or virtual keyboard key or any other interactive areas in this display, or the information is presented in any arbitrary, a.o. not-interactive display, wherein
this visual information is simultaneously presented immediately (directly) in front of the eyes as an imaginary image (virtual, aerial image, picture outside of a screen surface), for which aim
this a.m. visual information is transferred from the gadget in a looking device (monitor), which looking device is placed in glasses (spectacles) or in glasses extension, or in an other extension piece placed on a user's head, or in contact lenses (this looking device is further named below as “glasses looking device”),
and simultaneously an every position (spatial coordinates or 2D-coordinates in a display surface) of the a.m. working finger (or of the a.m. middle-stick, or only of their parts or only of tops or end points of the a.m. working finger or middle-stick) is located by a special device, and this information about this position is transferred from the gadget display in the glasses looking device in the real-time regime,
wherein a virtual image of this working finger or of this middle-stick is superimposed on a picture (image), which one is transferred from the gadget display in the glasses looking device (i.e. in other words, the two above-mentioned images (the first one—from the gadget display, and the second one—from the working finger (or middle-stick) position relative to the gadget display), are superimposed or put over each other in the same picture (image) in the glasses looking device,
wherein instead of a working finger (or of a middle-stick) one can present in a glasses looking device only a virtual image of it's top (or end point), a.o. in a form of a cursor (as for example cross or arrow),
wherein one can locate only two coordinates of the working finger top (or of a middle-stick top), in the plane of gadget display surface, without taking into account of a vertical distance between the working finger (or middle-stick) and gadget display surface,
wherein a.o. when executing of “enlargement” and “decreasing”—functions also an image of a second finger (or it's top) can temporary appear in a glasses looking device, after one has touched the gadget display by this finger, together with a working finger,
wherein a.o. a forefinger can be used as a working finger, and a thumb can be used as a second finger,
wherein a top of the working finger (or of the middle-stick) can be marked with any marker (a.o. magnetic marker, electrostatic marker, optic marker, etc.), to indicate a position of the a.m. top,
wherein instead of a single finger one can indicate several, up to 10 working fingers, locate their spatial positions, and use them to operate the system,
wherein one can a.o. generate the am. imaginary image only in front of one of the two eyes, for which aim a.o. one can transfer a visual information e.g. in a glasses extension, which one is placed on one from the two glasses lenses,
wherein the a.m. imaginary picture (image) a.o. can occupy either a whole field of vision or only a part of the whole field of vision, wherein a rest part of the field of vision remains for the surroundings observation; or a field of vision can be occupied by several imaginary pictures (images), which pictures (images) a.o. also can be transferred in the glasses looking device from several sources (a.o. from several gadgets).

In one embodiment example the visual information is not presented in the physical touch-sensitive gadget display, and the visual information is not operated by touching of the physical display, and instead:
every position (spatial coordinates or 2D-coordinates in some plane) of a working finger (or of several fingers, or of a middle-stick, or only of their parts, or of tops (end points) of the a.m. working finger or fingers or middle-stick) is located by means of a special device, and this information about this position is transferred in the glasses looking device in the real-time regime,
wherein a virtual image of this working finger or of these fingers or of this middle-stick) is superimposed on a picture (image), which one is transferred from the gadget in the glasses looking device (i.e. in other words, the two above-mentioned images (the first one—from the gadget, and the second one—from the working finger—(or middle-stick)—spatial position), are superimposed or put over each other in the same picture (image) in the glasses looking device,—this way the whole system is operated by the finger-controlled operations with the virtual (imagionary/appearing, aerial image-like, “in the air hanging”) interactive image.

In one embodiment example a virtual imaginary interactive image of a music keyboard is formed in the glasses looking device, which way one can play by means of the virtual keyboard keys the same way as one plays by means of real keyboard keys, a.o. by 10 fingers.

In one embodiment example a virtual imaginary interactive image of a PC-keyboard is formed in the glasses looking device, which way one can operate by means of the virtual keyboard keys the same way as one operates by means of real PC-keyboard keys, a.o. by 10 fingers.

In one embodiment example the a.m. special device (by means of which one the every position (spatial coordinates or 2D-coordinates in the display surface plane) of the a.m. working fingers (or of a single working finger, or of the a.m. middle-stick, or only of their parts or only of tops (end points) of the a.m. working fingers or middle-sticks) is located, and after that this information about this position is transferred in the a.m. glasses looking device on claim one in the real-time regime), is placed in the glasses looking device or in any kind of a head cover,
wherein the a.m. special device can be placed a.o. also on a wrist, or on a waist belt, or on any other part of the body, or on a table, or on any other kind of support or carrier near the working finger.

In one embodiment example the visual information is not presented in the physical touch-sensitive gadget display on claim one, and the visual information is not operated by touching of the a.m. physical display, and instead
the whole system is operated through a touchpad (a.o. the same way as it takes place in a laptop).

In one embodiment example the a.m. gadget is partially interlocked when one switches it off or when one charges it or after expiration of a previously adjustable time period, and when a user switches it on or for the further using the user places his eye in front of a videocamera, the visual information about the eye (a.o. about the eye-bottom) is electronically analysed by the gadget or by it's parts, and this information is compared with the previously provided information (picture), and after that the gadget is switched on as a completely able to work one, only if this new presented visual information corresponds to this previously provided information.

In one embodiment example a method for presentation and processing of information is executed, wherein either a stereo sound (stereo-acoustic sound) in headphones, or a picture (in particular a stereo 3D-picture), or both sound and picture, are presented, wherein
a) a perspective view (“aspect to the observer”) of a picture, or of a 3D-picture, is presented in a display (or in any kind of monitor/looking device) dependently on the user's head position (or on the head position changes, or on the head motions), or dependently on the user's eyes positions (or on the eyes positions changes, or on the eyes motions), i.e. when the user turns or bends his head, or when he changes a position of his head, the perspective view of a picture in a display or the perspective view of an imaginary picture in a looking device are also changed correspondently, or/and
b) a relationship between the acoustic parameters (a.o. a relationship among loudnesses) in a left and right headphones is changed dependently on the head position or eyes positions relative to an imaginary source of sound in a display (or in an aerial/virtual/appearing/imaginary/“in the air hanging” image), i.e. a.o. when an user turns or bends his head or he changes his head position, or when a virtual position of a virtual/imaginary sound source changes itself, a.o. also the sound loudnesses relationship in the left and right head phones is changed correspondently, the similar way as this change in sound loudnesses relationship in ears had to happen a.o. in the case of real head motions relative to real sound sources positions.

In one embodiment example of the method the additional vibrations are generated in a head cover (a.o. in head phones or in their separate parts), in particular in it's (head cover's) front or back parts, dependently on the circumstance, whether the virtual sound source is placed in front of or from behind of the user (head phones carrier).

In one embodiment example the device comprises a gadget, wherein the device further comprises:
1. device (means) to locate the user's eyes pupils position relative to the user's body, which device comprises:
   a) a device (means) to locate the position of eyes pupils relative to the head or face of the user. (It is enough to observe only one eye pupil, because the both eyes pupils move the same way. Therefore the device comprises a video camera, which one is placed only on a left, or only on a right part of glasses; or the device contains two videocameras, which are placed on the both left and right glasses clamps on the left and on the right on the left and right eyes; or the device comprises the two video cameras, which cameras are placed near the bridge of the nose, and the first one of these two cameras observes the motion of eye pupil in the left part of the right eye, and the second one of these two cameras observes the motion of eye pupil in the right part of the left eye, which way these two video cameras cover all motions of the both eyes pupils, because the eyes pupils motions are definitely dependent one from each other);
   b) a device (means) to observe the bendings (inclinations, nods), turns and shakings of a user's head relative to his body—in horizontal and vertical planes (a.o. e.g. by means of sensors or by means of a gyroscope);
   c) a device (means) to process the data from the mentioned above in the items (a) and (b) devices, and this way to locate and determine the user's eyes pupils positions relative to the user's body;
   d) a device (means) to collect and transfer the data from the mentioned above in the items (a) and (b) devices into the device on item (c);
   e) a device (means) to transfer the data from the device on item (c) into the device, which one is described in the next item (2) below;

2) a device (means) for processing the information from the mentioned above in the items (c), (d) and (e) devices (means), for generation of a display image, and for positioning of this image in front of the user dependently on the current momental positions of the user's eyes pupils relative to the user's body. This device can be placed in the glasses or in the eyes contact lenses of the user;

3) a device (means) to observe the user's finger and “interactive areas” in a virtual display, wherein this device can comprise two video cameras placed in the glasses clamps from the left side and from the right side, wherein when the finger (a.o. the right or the left forefinger) of the user is near the interactive area, an electronic lock-on (capture) of the finger happens, i.e. a determination that the finger is near the interactive area, and the device informs the user about it with a buzzer or by means of any kind of other known method (e.g. through enlargement of this interactive area or of some part or point of this area), and after that the user must confirm a choice (a.o. by a voice or by pressing on a contact device on a finger, a.o. by a thumb-finger, or the device generates a virtual part in (on) the virtual interactive area, and the user must grip this virtual part by two fingers (a.o. by the thumb and forefinger), which fact is fixed either by a placed on the glasses video camera (cameras), or the user has the correspondent contact-devices on his thumb and forefinger, wherein the pressing together of these two fingers is recognised as an Acknowledgement of the choice (which one analogically corresponds to the finger pressing on a gadget real display interactive area).

Instead of sensors, which determine a user's head position or motions (bending, turning, shaking), the device according to the item (1b) (s. above) can contain a gyroscope, and this way this device can determine all possible turns and motions of the user's head.

In one embodiment example of the method the a.m. gadget is partially interlocked when one switches it off, and when a user switches it on, he placed his eye in front of a videocamera, the visual information about the eye (a.o. about the eye-bottom) is electronically analysed by the gadget or by it's parts, and this information is compared with the previously provided information (picture), and after that the gadget is switched on as a completely able to work one, only if this new presented visual information corresponds to this previously provided information.

In one embodiment example of the method either a stereo sound (stereo-acoustic sound) in headphones, or a picture (in particular a stereo 3D-picture), or both sound and picture, are presented, and also
a) a perspective view (“aspect to the observer”) of a picture, or of a 3D-picture, is presented in a display (or in any kind of monitor/looking device) dependently on the user's head position, or dependently on the user's eyes positions, i.e. when the user turns or bends his head, or when he changes a position of his head, the perspective view of a picture in a display or the perspective view of an imaginary picture in a looking device are also changed correspondently, or/and
b) a relationship between the acoustic parameters (a.o. a relationship among loudnesses) in a left and right headphones is changed dependently on the head position or eyes positions relative to an imaginary source of sound in a display (or in an aerial/virtual/appearing/imaginary/“in the air hanging” image), i.e. a.o. when an user turns or bends his head or he changes his head position, or when a virtual position of a virtual/imaginary sound source changes itself, a.o. also the sound loudnesses relationship in the left and right head phones is changed correspondently, the similar way as this change in sound loudnesses relationship in the ears had to happen a.o. in the case of real head motions relative to real sound sources positions.

Furthermore, a.o. the following two special embodiment examples have to be described more detailed:
1) An usual case of an usual cinema or PC-game, where the case in point is a 2D or 3D videopresentation in a physical (real) display, and stereo audiopresentation in headphones. Here the relationship between the acoustic parameters (a.o. a relationship among loudnesses) in a left and right headphones has been changing when a virtual position of a virtual sound source in the display has been changing. For example, if a car (i.e. self-evidently a virtual picture (image) of a car) drives in a display from the left to the right;
2) In one embodiment example of the method the additional vibrations are generated in a head cover (a.o. in head phones or in their separate parts), in particular in it's (head cover's) front or back parts, dependently on the circumstance, whether the virtual sound source is placed in front or from behind of the user (head phones carrier).

In one embodiment example of the method the several, at least three, video cameras (webcameras) with the overlapping fields of vision are used to receive video signals. These at least 3 videocameras can provide altogether a.o. 360° field of vision. Also a device (a.o. a PC with a correspondent software), which one processes the provided by these cameras video signal information, is used. This device further represents this generally collected together from all cameras and processed information as a one picture in a display (or in an a.m. looking device) such way, that when reproducing picture in a display one can choose the different perspectives (points of view), independently on the direction of motion of the carrier of the cameras, and independently on the spatial orientation of the cameras in the current moment.

In one embodiment example of the method a stereo sound (stereo-acoustic sound) in headphones is presented, wherein
a) a perspective view (“aspect to the observer”) of a picture, a.o. of a 3D-picture) is presented in a display (or in a looking device), dependently on the user's head position (or on the head position changes, or on the head motions), or dependently on the user's eyes positions (or on the eyes positions changes, or on the eyes motions), i.e. when the user turns or bends his head, or when he changes a position of his head (and/or when he correspondently changes the position of his eyes), the perspective view of a picture in a display or the perspective view of an imaginary picture in a looking device are also changed correspondently,
   or/and
b) a relationship between the acoustic parameters (a.o. a relationship among loudnesses) in a left and right headphones is changed dependently on the head position or eyes positions relative to an imaginary source of sound in a real display of a computer (PC, laptop, Ipad, I-phone, smartphone etc.)
   or in an above-described aerial/virtual/appearing/imaginary/“in the air hanging” display), i.e. a.o. when an user turns or bends his head or he changes his head position, or when the eyes position is changed, i.e. when user directs his look at an other point in a display, or when a virtual position of a virtual/imaginary sound source changes itself, a.o. also the sound loudnesses relationship in the left and right head phones is changed correspondently, the similar way as this change in sound loudnesses relationship in ears had to happen a.o. in the case of real head motions (and a.o. consequently the eyes position changes) relative to real sound sources positions. This embodiment example can be executed both for an usual device with a real display (PC, laptop, Ipad, I-phone, smartphone etc.), and for an above-described looking device with a virtual (imaginary) display. The eyes positions can be observed by a video camera and analysed by a computer (gadget), as it is described in the U.S. Ser. No. 14/138,066, from Dec. 21, 2013.

In one embodiment example of the method the all above-described acoustic stereosound functions can be executed together with the functions, described in the US 14/138,066.

In one embodiment example of the method the all above-described functions, a.o. the picture(image)-presentation-functions can be executed together with the in U.S. Ser. No. 14/138,066 described functions.

(Abbreviation “a.m.” (from the German “o.g.”) means “above-mentioned”; Abbreviation “a.o.” (from the German “u.a.”) means “including among others” or “among other possibilities”).

What is claimed is:

1. A system or a device for interactive intercommunication with a book or with a e-book/kindle, comprising:
a) at least one book or e-book/kindle,
b) at least one computer with a display,
c) at least one identification device, i.e. a device for identification of an open page of a book (a.o. comprising a BO-webcamera, book pages with identification codes, and a computer, by means of which one the webcamera and the connected with this webcamera computer can identify this code and, consequently, the code-carrying page),
d) fastening means to fix the a.m. identification device (a.o. a BO-webcamera) to the book or to the user,
e) either: e-1) a device for recognition of separate points (places, areas) on the open page surface, which points (places, areas) are currently touched by an user with his finger or with a pointing agent (a.o. a role of this device can play a BO-webcamera, a.o. the same a.m. BO-webcamera, which one identifies the open page), wherein under the “open page” one means an open paper page of a book or a currently switched-on page of a e-book/kindle, wherein an e-book/kindle display itself is not interactive/ touch-sensitive, or
e-2) a device for recognition of position of the pupils of the user eyes relative to some definite points, areas or characters of the open page of a book (a.o. an UO-webcamera of a computer, i.e. an other camera, not the camera, which one is used for the identification of the open page and of the a.m. separate points (separate places/areas) of this page), a device for recognition, at what point (place, area) of the open page the user looks at, a device for analysing of the eyes pupils trajectories and consequently of the objects and priorities of the user's attention, and further consequently for analysing what kind of information, which one corresponds to these attention objects and priorities, have to be presented in the computer display (a role of these a.m. devices can play the a.m. computer with the both a.m. webcameras), or:
e-3) a device for recognition of position of the eyes pupils and facial expression/changes of facial expressions of the user (a.o. an UO-webcamera of a computer, i.e. an other camera, not the camera, which one is used for the identification of the open page and of the a.m. separate points (separate places/areas) of this page), and a device for recognition, at what point (place, area) of the open page the user looks at, as well as what kind of facial expression/changes of facial expressions has the user in a current moment, and consequently what kind of the information (which one corresponds to these pupils position and correspondent facial expression/changes of facial expressions) have to be presented in the computer display (a role of these a.m. devices can play the a.m. computer with the both a.m. webcameras), wherein one can use the both a.m. BO-webcamera and UO-webcamera (or many these webcameras) together;
f) website or other external data-carrier;
g) code-marking for general identification of a book;
h) code-markings for each page;
i) submarkings for matters inside pages.

2. A system or a device on claim 1, comprising an UO-webcamera and a computer, as well as the usual peripherals for an interactive communication of a user with a computer, wherein the system comprises a lie-detector, wherein a book proposes to an user the situations (textually, i.e. by means of words, or by means of pictures or both), and dependently on the user's reaction, and in accordance with the readings of the lie-detector, which are processed by computer, the computer chooses a variant of a further presentation on a computer display.

3. A system or a device on claim 2,
wherein instead of (or additionally to) the lie-detector the system comprises an encephalograph, a myograph, or any other medical-diagnostic equipment, which equipment executes a reading of the current medical-biological parameters (in particular finally of an emotional state) of an user, watches these data and passes these data into the computer, after that these data are processed by a computer program, and, dependently on the results of this processing, the computer choose a variant of further presentations on a display.

4. A system or a device on claim 1,
comprising an additional display, which display is placed on the (in the) glasses/spectacles, or on the (in the) contact lens (or lenses), and besides, in particular, this display is connected with a mobile telephone device electrically or electromagnetically by a wire or wireless, wherein the signals, which are normally causing a real image on a display of a smartphone/mobile telephone, are converted in an image, in particular in a virtual image (appearing/imaginary/“in the air hanging” image), which one appears directly (immediate) in front of the user's eyes, in particular this displayed matter (image) is created/formed in the lenses of glasses (spectacles), or in the lenses cover pieces, or only in one of the two lenses cover pieces, or in the eyes contact lenses, or only in one of the two eyes contact lenses.

5. A system or a device on claim 4,
comprising two displays, each one for one of the eyes, and also it comprises a.o a hardware and software for a creating of 3-D images or for a presenting of stereopictures.

6. Method of intercommunication with a book (a.o. with a “hard-cover”-book, paper-cover/soft-cover book, comics, journal, or with any other kind of printed edition, or with a e-book/kindle),
wherein the book comprises one or several UO-webcameras and BO-webcameras or optionally also others, usual kinds of peripherals for interactive communication of a user with a computer, wherein
the position of the pupils of the user's eyes relative to a book page (or relative to a page of a e-book/kindle), is observed by means of the UO-webcamera or UO-webcameras, with or without a simultaneous observation of the eyes pupils dimensions or of the eyes pupils dimensions changes, inclusively an information
about trajectories of the eyes pupils motion relative to the book page through the places, where the user fixes his look, as well as
about the time periods of these pupils motions and time periods of these look fixations are observed, i.e. a.o. an information about the user's attention concentration objects is observed,
after that this information, together with the observation information from the BO-webcamera (webcameras) is transferred in the computer, is converted in the digital form, analysed and processed, and then, dependently on the results, the computer presents on it's display or in an audio output a current comment or information, which one corresponds to the results of user's reaction to the current book page or to the current places in this book page, wherein the computer takes these comments or information from a website, which one corresponds to this book (or to this book among other books), or from any other kind of memory & processing resource, for example from a computer itself.

7. Method on claim 6,
wherein instead of (or additionally to) the observing of the positions and dimensions of user's eyes pupils, the computer also observes by a BO-webcamera a finger position of the user, which user points by his finger to different places in a book page, and this way he calls for correspondent comments or information from the computer or a.o. from the website, wherein the user can both point to different places in a book page, and circle (outline) some areas in a book page by his finger, or he can execute other previously provided finger motions, which correspond to definite reactions of the computer.

8. Method on claim 6,
wherein
an user's facial expression (state of face muscles) or the changes of the facial expression (face muscles tensions), a.o. eye—or eye's pupils muscle tensions of a user are watched by a web camera or by a UO-webcamera or by UO-webcameras, then this information is transferred in the computer, is converted in the digital (electronic) form, is analysed and processed, and after that, dependently on the results of these analyse and processing, the computer changes a displayed matter (picture) on the computer's display in accordance with this information, or the computer changes the currently acting reactions of the computer game on the actions of the user, wherein an each reaction of a user corresponds to that book page or to that place in a page, whereto the user currently or oftly looks at (i.e. where the user's eyes pupils are directed to).

9. Method on claim 6,
comprising a creation of a visual information in a display of a smartphone (mobile telephone), wherein these signals, which are normally causing a real image on a display of a smartphone/mobile telephone, are converted in an image, in particular in a virtual image (appearing/imaginary/“in the air hanging” image), which one appears directly (immediate) in front of the user's eyes, in particular this displayed matter (image) is created/formed in the lenses of glasses (spectacles), or in the lenses cover pieces, or only in one of the two lenses cover pieces, or in the eye contact lenses, or only in one of the two eye contact lenses.

10. Method for presentation and processing of information, wherein a visual information is presented in a gadget display, a.o. in an interactive touchable (touch-sensitive) gadget display (a.o. of a smartphone or of an I-pad etc.), and this visual information is operated by a finger (this finger will be further named below as a “working finger”), or this visual information is operated by a mediator (middle-body), for example by a stick or needle (further named below as a “middle-stick”), for which purpose one touch by the a.m. working finger or by the a.m. middle-stick a correspondent virtual picture (a.o. an icon—i.e. a graphic symbol in a display) or a virtual keyboard key or any other interactive areas in this display, or the information is presented in any arbitrary, a.o. not-interactive display,
wherein
this visual information is simultaneously presented immediately (directly) in front of the eyes as an imaginary image (virtual, aerial image, picture outside of a screen surface), for which aim
this a.m. visual information is transferred from the gadget in a looking device (monitor), which looking device is placed in glasses (spectacles) or in glasses extension, or in an other extension piece placed on a user's head, or in contact lenses (this looking device is further named below as “glasses looking device”),
and simultaneously an every position (spatial coordinates or 2D-coordinates in a display surface) of the a.m. working finger (or of the a.m. middle-stick, or only of their parts or only of tops or end points of the a.m. working finger or middle-stick) is located by a special device, and this information about this position is transferred in the glasses looking device in the real-time regime,
wherein a virtual image of this working finger or of this middle-stick is superimposed on a picture (image), which one is transferred from the gadget display in the glasses looking device (i.e. with another words, the two above-mentioned images (the first one—from the gadget display, and the second one—from the working finger (or middle-stick) position relative to the gadget display), are superimposed or put over each other in the same picture (image) in the glasses looking device,
wherein instead of a working finger (or of a middle-stick) one can present in a glasses looking device only a virtual image of it's top (or end point), a.o. in a form of a cursor (as for example cross or arrow),
wherein one can locate only two coordinates of the working finger top (or of a middle-stick top), in the plane of gadget display surface, without taking into account of a vertical distance between the working finger (or middle-stick) and gadget display surface,
wherein a.o. when executing of “enlargement” and “decreasing”—functions also an image of a second finger (or it's top) can temporary appear in a glasses looking device, after one has touched the gadget display by this finger, together with a working finger,
wherein a.o. a forefinger can be used as a working finger, and a thumb can be used as a second finger,
wherein a top of the working finger (or of the middle-stick) can be marked with any marker (a.o. magnetic marker, electrostatic marker, optic marker, etc.), to indicate a position of the a.m. top,
wherein instead of a single finger one can indicate several, up to 10 working fingers, locate their spatial positions, and use them to operate the system,
wherein one can a.o. generate the a.m. imaginary image only in front of one of the two eyes, for which aim a.o. one can transfer a visual information e.g. in a glasses extension, which one is placed on one from the two glasses lenses,
wherein the a.m. imaginary picture (image) a.o. can occupy either a whole field of vision or only a part of the whole field of vision, wherein a rest part of the field of vision remains for the surroundings observation; or a field of vision can be occupied by several imaginary pictures (images), which pictures (images) a.o. also can be transferred in the glasses looking device from several sources (a.o. from several gadgets).

11. Method on claim 10,
wherein the visual information is not presented in the physical a.m. touch-sensitive gadget display, and the visual information is not operated by touching of the a.m. physical display, and instead:
every position (spatial coordinates or 2D-coordinates in some plane) of a working finger (or of several fingers, or of a middle-stick, or only of their parts, or of tops (end points) of the a.m. working finger or fingers or middle-stick) is located by means of a special device, and this information about this position is transferred in the glasses looking device in the real-time regime,
wherein a virtual image of this working finger or of these fingers or of this middle-stick) is superimposed on a picture (image), which one is transferred from the gadget in the glasses looking device (i.e. with another words, the two above-mentioned images (the first one—from the gadget, and the second one—from the working finger—(or middle-stick)—spatial position), are superimposed or put over each other in the same picture (image) in the glasses looking device,
this way the whole system is operated by the finger-controlled operations with the virtual (imaginary/appearing, aerial image-like, “in the air hanging”) interactive image.

12. Method on claim 11, wherein a virtual imaginary interactive image of a music keyboard is formed in the glasses looking device, which way one can play by means of the virtual keyboard keys the same way as one plays by means of real keyboard keys, a.o. by 10 fingers.

13. Method on claim 11, wherein a virtual imaginary interactive image of a PC-keyboard is formed in the glasses looking device, which way one can operate by means of the virtual keyboard keys the same way as one operates by means of real PC-keyboard keys, a.o. by 10 fingers.

14. Method on claim 10,
wherein the special device on claim 10
(by means of which one the every position (spatial coordinates or 2D-coordinates in the display surface plane) of the a.m. working fingers (or of a single working finger, or of the a.m. middle-stick, or only of their parts or only of tops (end points) of the a.m. working fingers or middle-sticks) is located, and after that this information about this position is transferred in the a.m. glasses looking device in the real-time regime),
is placed in the glasses looking device or in any kind of a head cover,
wherein the a.m. special device can be placed a.o. also on a wrist, or on a waist belt, or on any other part of the body, or on a table, or on any other kind of support or carrier near the working finger.

15. Method on claim 11,
wherein the special device (by means of which one the every position (spatial coordinates or 2D-coordinates in the display surface plane) of the a.m. working fingers (or of a single working finger, or of the a.m. middle-stick, or only of their parts or only of tops (end points) of the a.m. working fingers or middle-sticks) is located, and after that this information about this position is transferred in the a.m. glasses looking device in the real-time regime), is placed in the glasses looking device or in any kind of a head cover,
wherein the a.m. special device can be placed a.o. also on a wrist, or on a waist belt, or on any other part of the body, or on a table, or on any other kind of support or carrier near the working finger.

16. Method on claim 10,
wherein the visual information is not presented in the a.m. physical touch-sensitive gadget display, and the visual information is not operated by touching of the a.m. physical display, and instead
the whole system is operated through a touchpad (a.o. the same way as it takes place in a laptop).

17. Method on claim 10, wherein the a.m. gadget (or any kind of electronic device) is partially interlocked when one switches it off, and when a user switches it on, he places his eye in front of a videocamera, the visual information about the eye (a.o. about the eye-bottom) is electronically analysed by the gadget or by it's parts, and this information is compared with the previously provided information (picture), and after that the gadget is switched on as a completely able to work one, only if this new presented visual information corresponds to this previously provided information.

18. Method for presentation and processing of information, in particular also on claim 11, wherein either a stereo sound (stereo-acoustic sound) in headphones, or a picture (in particular a stereo 3D-picture), or both sound and picture, are presented, wherein
a) a perspective view (“aspect to the observer”) of a picture, or of a 3D-picture, is presented in a display (or in any kind of monitor/looking device) dependently on the user's head position (or on the head position changes, or on the head motions), or dependently on the user's eyes positions (or on the eyes positions changes, or on the eyes motions), i.e. when the user turns or bends his head, or when he changes a position of his head, the perspective view of a picture in a display or the perspective view of an imaginary picture in a looking device are also changed correspondently, or/and
b) a relationship between the acoustic parameters (a.o. a relationship among loudnesses) in a left and right headphones is changed dependently on the head position or eyes positions relative to an imaginary source of sound in a display (or in an aerial/virtual/appearing/imaginary/“in the air hanging” image), i.e. a.o. when an user turns or bends his head or he changes his head position, or when a virtual position of a virtual/imaginary sound source changes itself, a.o. also the sound loudnesses relationship in the left and right head phones is changed correspondently, the similar way as this change in sound loudnesses relationship in ears had to happen a.o. in the case of real head motions relative to real sound sources positions.

19. Method on claim 18, wherein additional vibrations are generated in a head cover (a.o. in head phones or in their separate parts), in particular in it's (head cover's) front or back parts, dependently on the circumstance, whether the virtual sound source is placed in front or from behind of the user (head phones carrier).

20. Device for execution of the method on claim 11, comprising a gadget, wherein the device also comprises:
1) device (means) to locate the user's eyes pupils position relative to the user's body, which device comprises:
a) a device (means) to locate the position of eyes pupils relative to the head or face of the user (wherein it is enough to observe only one eye pupil, because the both eyes pupils move the same way; and therefore the device comprises a video camera, which one is placed only on a left, or only on a right part of glasses; or the device contains two videocameras, which are placed on the both left and right glasses clamps on the left and on the right on the left and right eyes; or the device comprises the two video cameras, which cameras are placed near the bridge of the nose, and the first one of these two cameras observes the motion of eye pupil in the left part of the right eye, and the second one of these two cameras observes the motion of eye pupil in the right part of the left eye, which way these two video cameras cover all motions of the both eyes pupils, because the eyes pupils motions are definitely dependent one from each other);
b) a device (means) to observe the bendings (inclinations, nods), turns and shakings of a user's head relative to his body—in horizontal and vertical planes (a.o. e.g. by means of sensors or by means of a gyroscope);
c) a device (means) to process the data from the mentioned above in the items (a) and (b) devices, and this way to locate and determine the user's eyes pupils positions relative to the user's body;
d) a device (means) to collect and transfer the data from the mentioned above in the items (a) and (b) devices into the device on item (c);
e) a device (means) to transfer the data from the device on item (c) into the device, which one is described in the next item (2) below;
2) a device (means) for processing the information from the mentioned above in the items (c), (d) and (e) devices (means), for generation of a display image, and for positioning of this image in front of the user dependently on the current momental positions of the user's eyes pupils relative to the user's body, wherein this device can be placed in the glasses or in the eyes contact lenses of the user;
3) a device (means) to observe the user's finger and “interactive areas” in a virtual display, wherein this device can comprise two video cameras placed in the glasses clamps from the left side and from the right side, wherein when the finger (a.o. the right or the left forefinger) of the user is near the interactive area, an electronic lock-on (capture) of the finger happens, i.e. a determination that the finger is near the interactive area, and the device informs the user about it with a buzzer or by means of any kind of other known method (e.g. through enlargement of this interactive area or of some part or point of this area), and after that the user must confirm a choice (a.o. by a voice or by pressing on a contact device on a finger, a.o. by a thumb-finger, or the device generates a virtual part in (on) the virtual interactive area, and the user must grip this virtual part by two fingers (a.o. by the thumb and forefinger), which fact is fixed either by a placed on the glasses video camera (cameras), or the user has the correspondent contact-devices on his thumb and forefinger, wherein the pressing together of these two fingers is recognised as an Acknowledgement of the choice (which one analogically corresponds to the finger pressing on a gadget real display interactive area).
